(19) United States
(12) Patent Application Publication
(10) Pub. No.: US 2016/0099416 A1
(43) Pub. Date: Apr. 7, 2016

(54) MATERIAL FOR ORGANIC ELECTROLUMINESCENT DEVICE AND ORGANIC ELECTROLUMINESCENT DEVICE INCLUDING THE SAME

(71) Applicant: SAMSUNG DISPLAY CO., LTD., Yongin-City (KR)

(72) Inventor: Hiroaki ITOI, Yokohama (JP)

(21) Appl. No.: 14/790,703

(22) Filed: Jul. 2, 2015

(30) Foreign Application Priority Data
Oct. 6, 2014 (JP) 2014-205669

(51) Int. Cl. H01L 51/00; C07D 498/06

(52) U.S. Cl. CPC H01L 51/0061; C07D 498/06; H01L 51/0071; H01L 51/5012

(57) ABSTRACT

A compound for an organic electroluminescent device is represented by the following General Formula (1). X in General Formula (1) is selected from the following General Formulae (2), (3) and (4).

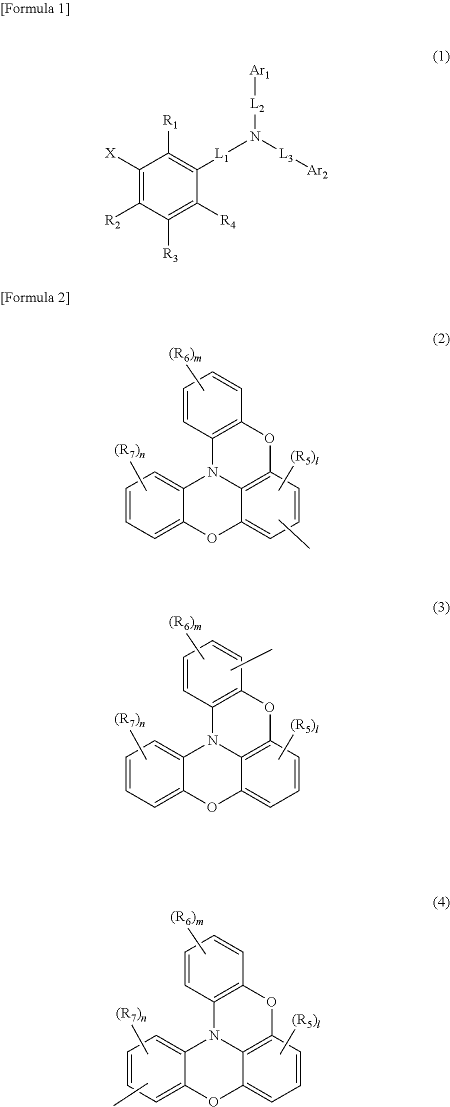

### CROSS-REFERENCE TO RELATED APPLICATION

Japanese Patent Application No. 2014-205669, filed on Oct. 6, 2014, in the Japanese Patent Office, and entitled: “Material For Organic Electroluminescent Device and Organic Electroluminescent Device Including The Same,” is incorporated by reference herein in its entirety.

### BACKGROUND

1. Field

Embodiments relate to a material for an organic electroluminescent device and an organic electroluminescent device including the same.

2. Description of the Related Art

Recently, the development of an organic electroluminescence display (organic EL display) as an image display is being actively conducted. The organic EL display is different from a liquid crystal display and is a self-luminescent display realizing display from a luminescent material including an organic compound in an emission layer by recombining holes and electrons injected from an anode and a cathode.

As an organic electroluminescent device (organic EL device), an organic device may include, e.g. an anode, a hole transport layer disposed on the anode, an emission layer disposed on the hole transport layer, an electron transport layer disposed on the emission layer and a cathode disposed on the electron transport layer. Holes are injected from the anode, and the injected holes move via the hole transport layer and are injected into the emission layer. Electrons are injected from the cathode, and the injected electrons move via the electron transport layer and are injected into the emission layer. The holes and electrons injected into the emission layer recombine to generate excitons in the emission layer. The organic EL device emits light using light generated by the radiation deactivation of the excitons. Various modifications to the organic IL device are possible.

### SUMMARY

Embodiments are directed to a compound for an organic electroluminescent (EL) device, the compound being represented by the following General Formula (1), wherein X in General Formula (1) is selected from the following General Formulae (2), (3), and (4):

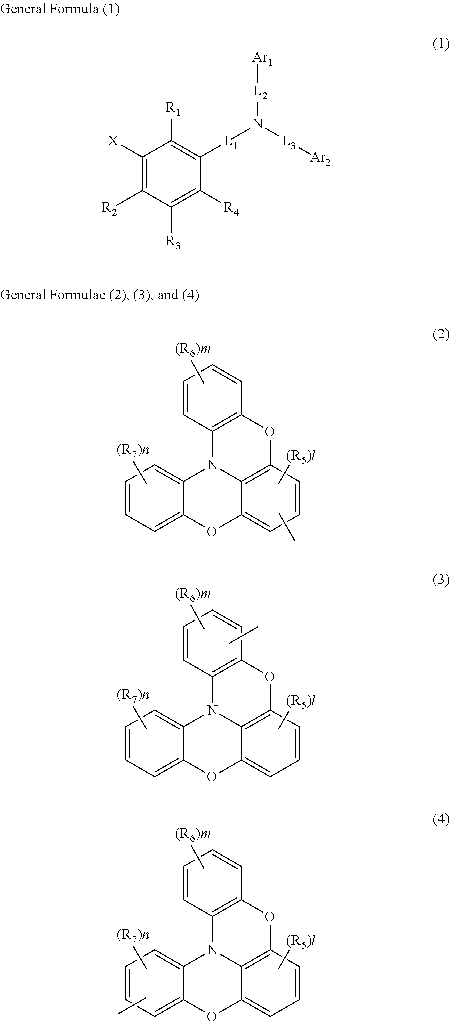

wherein in General Formula (1), R₁, R₂, R₃ and R₄ are independently a substituted or unsubstituted aryl group having 6 to 30 ring carbon atoms, a substituted or unsubstituted heteroaryl group having 1 to 30 ring carbon atoms, an alkyl group having 1 to 15 carbon atoms, a silyl group, a halogen atom, a hydrogen atom or a deuterium atom, L₁, L₂ and L₃ are independently a divalent group selected from a substituted or unsubstituted alkylene group, aralkylene group, arylene group and heteroarylene group, or a direct linkage, Ar₁ and Ar₂ are independently a substituted or unsubstituted aryl group having 6 to 30 ring carbon atoms or a substituted or unsubstituted heteroaryl group having 1 to 30 ring carbon atoms, and in General Formulae (2), (3), and (4), R₅, R₆ and R₇ are independently a substituted or unsubstituted aryl group having 6 to 30 ring carbon atoms, a substituted or unsubstituted heteroaryl group having 1 to 30 ring carbon atoms, an alkyl group having 1 to 15 carbon atoms, a silyl group, a halogen atom, a hydrogen atom or a deuterium atom, and l, n and m are independently an integer from 0 to 2.

X in General Formula (1) may be represented by General Formula (2).

Ar₁ and Ar₂ in the above General Formula (1) may be a substituted or unsubstituted aryl group having 6 to 30 ring carbon atoms.

Multiple adjacent R₁, R₂, R₃ and R₄ and/or R₅, R₆ and R₇ may be combined to form a saturated or unsaturated ring.

Embodiments are also directed to an organic electroluminescent (EL) device including a compound for an organic EL device in a layer of stacking layers between an emission layer and an anode. The compound for an organic EL device is represented by the following General Formula (1), and X in the above General Formula (1) is selected from the following General Formulae (2), (3) and (4):

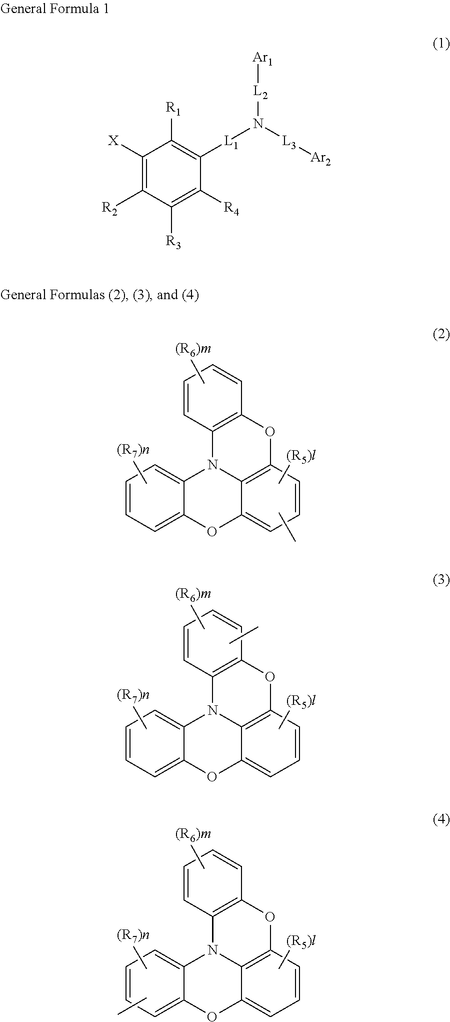

In General Formula (1), R₁, R₂, R₃ and R₄ are independently a substituted or unsubstituted aryl group having 6 to 30 ring carbon atoms, a substituted or unsubstituted heteroaryl group having 1 to 30 ring carbon atoms, an alkyl group having 1 to 15 carbon atoms, a silyl group, a halogen atom, a hydrogen atom or a deuterium atom, L₁, L₂ and L₃ are independently a divalent group selected from a substituted or unsubstituted alkylene group, aralkylene group, arylene group and heteroarylene group, or a direct linkage, and Ar₁ and Ar₂ are independently a substituted or unsubstituted aryl group having 6 to 30 ring carbon atoms or a substituted or unsubstituted heteroaryl group having 1 to 30 ring carbon atoms. In General Formula (2), (3), and (4), R₅, R₆ and R₇ are independently a substituted or unsubstituted aryl group having 6 to 30 ring carbon atoms, a substituted or unsubstituted heteroaryl group having 1 to 30 ring carbon atoms, an alkyl group having 1 to 15 carbon atoms, a silyl group, a halogen atom, a hydrogen atom or a deuterium atom, and l, n and m are independently an integer from 0 to 2.

X in General Formula (1) may be represented by General Formula (2).

Ar₁ and Ar₂ in the above General Formula (1) may be a substituted or unsubstituted aryl group having 6 to 30 ring carbon atoms.

Multiple adjacent R₁, R₂, R₃ and R₄ and/or R₅, R₆ and R₇ may be combined to form a saturated or unsaturated ring.

The compound for an organic EL device may be at least one of compounds 1 to 60 disclosed in the detailed description, below.

### BRIEF DESCRIPTION OF THE DRAWINGS

Features will become apparent to those of skill in the art by describing in detail exemplary embodiments with reference to the attached drawings in which:

FIG. 1 illustrates a schematic diagram showing an organic electroluminescent device according to an embodiment.

### DETAILED DESCRIPTION

Example embodiments will now be described more fully hereinafter with reference to the accompanying drawings; however, they may be embodied in different forms and should not be construed as limited to the embodiments set forth herein. Rather, these embodiments are provided so that this disclosure will be thorough and complete, and will fully convey exemplary implementations to those skilled in the art.

In the drawing figures, the dimensions of layers and regions may be exaggerated for clarity of illustration. It will also be understood that when a layer or element is referred to as being “on” another layer or substrate, it can be directly on the other layer or substrate, or intervening layers may also be present. Further, it will be understood that when a layer is referred to as being “between” two layers, it can be the only layer between the two layers, or one or more intervening layers may also be present. Like reference numerals refer to like elements throughout.

According to embodiments, an amorphous property may be maintained, and high mobility of holes, low driving voltage and high emission efficiency may be realized by introducing a benzoxazinophenoxazine part with high planarity at the meta position of a phenylene group with small conjugation effect in an amine having high amorphous property and high hole mobility.

Hereinafter, a material (i.e., a compound) for an organic EL device and an organic EL device including the same will be explained referring to attached drawing.

The material for an organic EL device may include an amine derivative represented by the following General Formula (1).

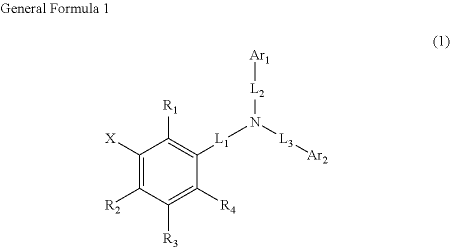

In General Formula (1), X is selected from benzoxazinophenoxazine moieties represented by the following General Formulae (2), (3) and (4).

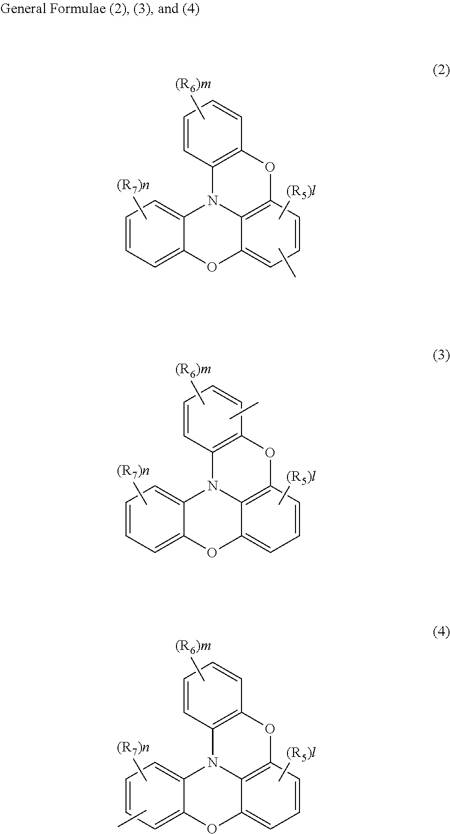

In General Formula (1), R₁, R₂, R₃ and R₄ are independently a substituted or unsubstituted aryl group having 6 to 30 ring carbon atoms, a substituted or unsubstituted heteroaryl group having 1 to 30 ring carbon atoms, an alkyl group having 1 to 15 carbon atoms, a silyl group, a halogen atom, a hydrogen atom or a deuterium atom. L₁, L₂ and L₃ are independently a divalent group selected from the group consisting of a substituted or unsubstituted alkylene group, aralkylene group, arylene group and heteroarylene group, or a direct linkage. Ar₁ and Ar₂ are a substituted or unsubstituted aryl group having 6 to 30 ring carbon atoms or a substituted or unsubstituted heteroaryl group having 1 to 30 ring carbon atoms. In addition, multiple adjacent R₁, R₂, R₃, R₄, R₅, R₆ and R₇ may combine to form a saturated or unsaturated ring.

The substituted or unsubstituted aryl group having 6 to 30 ring carbon atoms used in R₁, R₂, R₃ and R₄ may independently include a phenyl group, a naphthyl group, an anthracenyl group, a phenanthryl group, a biphenyl group, a terphenyl group, a quaterphenyl group, a quinquephenyl group, a sexiphenyl group, a fluorenyl group, a triphenylene group, a biphenylene group, a pyrenyl group, a benzofluoranthenyl, a chrysenyl group, etc.

The substituted or unsubstituted heteroaryl group having 1 to 30 ring carbon atoms used in R₁, R₂, R₃ and R₄ may independently include a benzothiazolyl group, a thiophenyl group, a thienothiophenyl group, a thienothienothiophenyl group, a benzothiophenyl group, a benzofuryl group, a dibenzothiophenyl group, a dibenzofuryl group, a N-arylcarbazolyl group, a N-heteroarylcarbazolyl group, a N-alkylcarbazolyl group, a phenoxazyl group, a phenothiazyl group, a pyridyl group, a pyrimidyl group, a triazile group, a quinolinyl group, a quinoxalyl group, etc.

The alkyl group having 1 to 15 carbon atoms used in R₁-R₄ may include a methyl group, an ethyl group, a propyl group, an isopropyl group, a n-butyl group, a s-butyl group, an isobutyl group, a t-butyl group, a n-pentyl group, a n-hexyl group, a n-heptyl group, a n-octyl group, a hydroxymethyl group, a 1-hydroxyethyl group, a 2-hydroxyethyl group, a 2-hydroxyisobutyl group, a 1,2-dihydroxyethyl group, a 1,3-dihydroxyisopropyl group, a 2,3-dihydroxy-t-butyl group, a 1,2,3-trihydroxypropyl group, a chloromethyl group, a 1-chloroethyl group, a 2-chloroethyl group, a 2-chloroisobutyl group, a 1,2-dichloroethyl group, a 1,3-dichloroisopropyl group, a 2,3-dichloro-t-butyl group, a 1,2,3-trichloropropyl group, a bromomethyl group, a 1-bromoethyl group, a 2-bromoethyl group, a 2-bromoisobutyl group, a 1,2-dibromoethyl group, a 1,3-dibromoisopropyl group, a 2,3-dibromo-t-butyl group, a 1,2,3-tribromopropyl group, an iodomethyl group, a 1-iodoethyl group, a 2-iodoethyl group, a 2-iodoisobutyl group, a 1,2-diiodoethyl group, a 1,3-diiodoisopropyl group, a 2,3-diiodo-t-butyl group, a 1,2,3-triiodopropyl group, an aminomethyl group, a 1-aminoethyl group, a 2-aminoethyl group, a 2-aminoisobutyl group, a 1,2-diaminoethyl group, a 1,3-diaminoisopropyl group, a 2,3-diamino-t-butyl group, a 1,2,3-triaminopropyl group, a cyanomethyl group, a 1-cyanoethyl group, a 2-cyanoethyl group, a 2-cyanoisobutyl group, a 1,2-dicyanoethyl group, a 1,3-dicyanoisopropyl group, a 2,3-dicyano-t-butyl group, a 1,2,3-tricyanopropyl group, a nitromethyl group, a 1-nitroethyl group, a 2-nitroethyl group, a 2-nitroisobutyl group, a 1,2-dinitroethyl group, a 1,3-dinitroisopropyl group, a 2,3-dinitro-t-butyl group, a 1,2,3-trinitropropyl group, a cyclopropyl group, a cyclobutyl group, a cyclopentyl group, a cyclohexyl group, a 4-methylcyclohexyl group, a 1-adamantyl group, a 2-adamantyl group, a 1-norbornyl group, a 2-norbornyl group, etc.

The substituted or unsubstituted aryl group having 6 to 30 ring carbon atoms used as Ar₁ and Ar₂ may include a phenyl group, a naphthyl group, an anthracenyl group, a phenanthryl group, a biphenyl group, a terphenyl group, a quaterphenyl group, a quinquephenyl group, a sexiphenyl group, a fluorenyl group, a triphenylene group, a biphenylene group, a pyrenyl group, a benzofluoranthenyl group, a chrysenyl group, etc.

The hetero aryl group having 1 to 30 ring carbon atoms used as Ar₁ and Ar₂ may include a benzothiazolyl group, a thiophenyl group, a thienothiophenyl group, a thienothienothiophenyl group, a benzothiophenyl group, a benzofuryl group, a dibenzothiophenyl group, a dibenzofuryl group, a N-arylcarbazolyl group, a N-heteroarylcarbazolyl group, a N-alkylcarbazolyl group, a phenoxazyl group, a phenothiazyl group, a pyridyl group, a pyrimidyl group, a triazile group, a quinolinyl group, a quinoxalyl group, etc.

The substituted or unsubstituted aryl group having 6 to 30 ring carbon atoms used in R₅-R₇ in General Formulae (2), (3), and (4) may include a phenyl group, a naphthyl group, an anthracenyl group, a phenanthryl group, a biphenyl group, a terphenyl group, a quaterphenyl group, a quinquephenyl group, a sexiphenyl group, a fluorenyl group, a triphenylene group, a biphenylene group, a pyrenyl group, a benzofluoranthenyl, a chrysenyl group, etc. In addition, multiple adjacent R₅-R₇ may combine and form a saturated or unsaturated ring.

The substituted or unsubstituted heteroaryl group having 1 to 30 ring carbon atoms used in R₅, R₆ and R₇ may include a benzothiazolyl group, a thiophenyl group, a thienothiophenyl group, a thienothienothiophenyl group, a benzothiophenyl group, a benzofuryl group, a dibenzothiophenyl group, a dibenzofuryl group, a N-arylcarbazolyl group, a N-heteroarylcarbazolyl group, a N-alkylcarbazolyl group, a phenoxazyl group, a phenothiazyl group, a pyridyl group, a pyrimidyl group, a triazile group, a quinolinyl group, a quinoxalyl group, etc. without limitation.

In addition, the alkyl group having 1 to 15 carbon atoms used in R₅, R₆ and R₇ may include a methyl group, an ethyl group, a propyl group, an isopropyl group, a n-butyl group, a s-butyl group, an isobutyl group, a t-butyl group, a n-pentyl group, a n-hexyl group, a n-heptyl group, a n-octyl group, a hydroxymethyl group, a 1-hydroxyethyl group, a 2-hydroxyethyl group, a 2-hydroxyisobutyl group, a 1,2-dihydroxyethyl group, a 1,3-dihydroxyisopropyl group, a 2,3-dihydroxy-t-butyl group, a 1,2,3-trihydroxypropyl group, a chloromethyl group, a 1-chloroethyl group, a 2-chloroethyl group, a 2-chloroisobutyl group, a 1,2-dichloroethyl group, a 1.3-dichloroisopropyl group, a 2,3-dichloro-t-butyl group, a 1,2,3-trichloropropyl group, a bromomethyl group, a 1-bromoethyl group, a 2-bromoethyl group, a 2-bromoisobutyl group, a 1,2-dibromoethyl group, a 1,3-dibromoisopropyl group, a 2,3-dibromo-t-butyl group, a 1,2,3-tribromopropyl group, an iodomethyl group, a 1-iodoethyl group, a 2-iodoethyl group, a 2-iodoisobutyl group, a 1,2-diiodoethyl group, a 1,3-diiodoisopropyl group, a 2,3-diiodo-t-butyl group, a 1,2,3-triiodopropyl group, an aminomethyl group, a 1-aminoethyl group, a 2-aminoethyl group, a 2-aminoisobutyl group, a 1,2-diaminoethyl group, a 1,3-diaminoisopropyl group, a 2,3-diamino-t-butyl group, a 1,2,3-triaminopropyl group, a cyanomethyl group, a 1-cyanoethyl group, a 2-cyanoethyl group, a 2-cyanoisobutyl group, a 1,2-dicyanoethyl group, a 1,3-dicyanoisopropyl group, a 2,3-dicyano-t-butyl group, a 1,2,3-tricyanopropyl group, a nitromethyl group, a 1-nitroethyl group, a 2-nitroethyl group, a 2-nitroisobutyl group, a 1,2-dinitroethyl group, a 1,3-dinitroisopropyl group, a 2,3-dinitro-t-butyl group, a 1,2,3-trinitropropyl group, a cyclopropyl group, a cyclobutyl group, a cyclopentyl group, a cyclohexyl group, a 4-methylcyclohexyl group, a 1-adamantyl group, a 2-adamantyl group, a 1-norbornyl group, 2-norbornyl group, etc.

In an implementation, X in General Formula (1) may be the benzoxazinophenoxazine part represented by General Formula (2). In the resulting compound of General Formula (1), the benzoxazinophenoxazine part represented by General Formula (2) is introduced to the meta position of a phenylene group of an amine derivative. Accordingly, a driving voltage may be decreased and emission efficiency may be increased for the organic EL device.

In the material for an organic EL device, Ar₁ and Ar₂ may be a substituted or unsubstituted aryl group having 6 to 30 ring carbon atoms. By introducing the substituted or unsubstituted aryl group having 6 to 30 ring carbon atoms in Ar₁ and Ar₂, a driving voltage may be decreased and emission efficiency may be increased for the organic EL device.

The material for an organic EL device may be obtained by introducing the benzoxazinophenoxazine part with high planarity to the meta position of the phenylene group with small conjugation effect to an amine having a high amorphous property and high hole mobility. The material may maintain the amorphous property and provide high hole mobility, low driving voltage and high emission efficiency. For example, remarkable effects may be obtained in a blue emission region.

An example of the material for an organic EL device may be a compound represented by one of the following formulae 1 to 6.

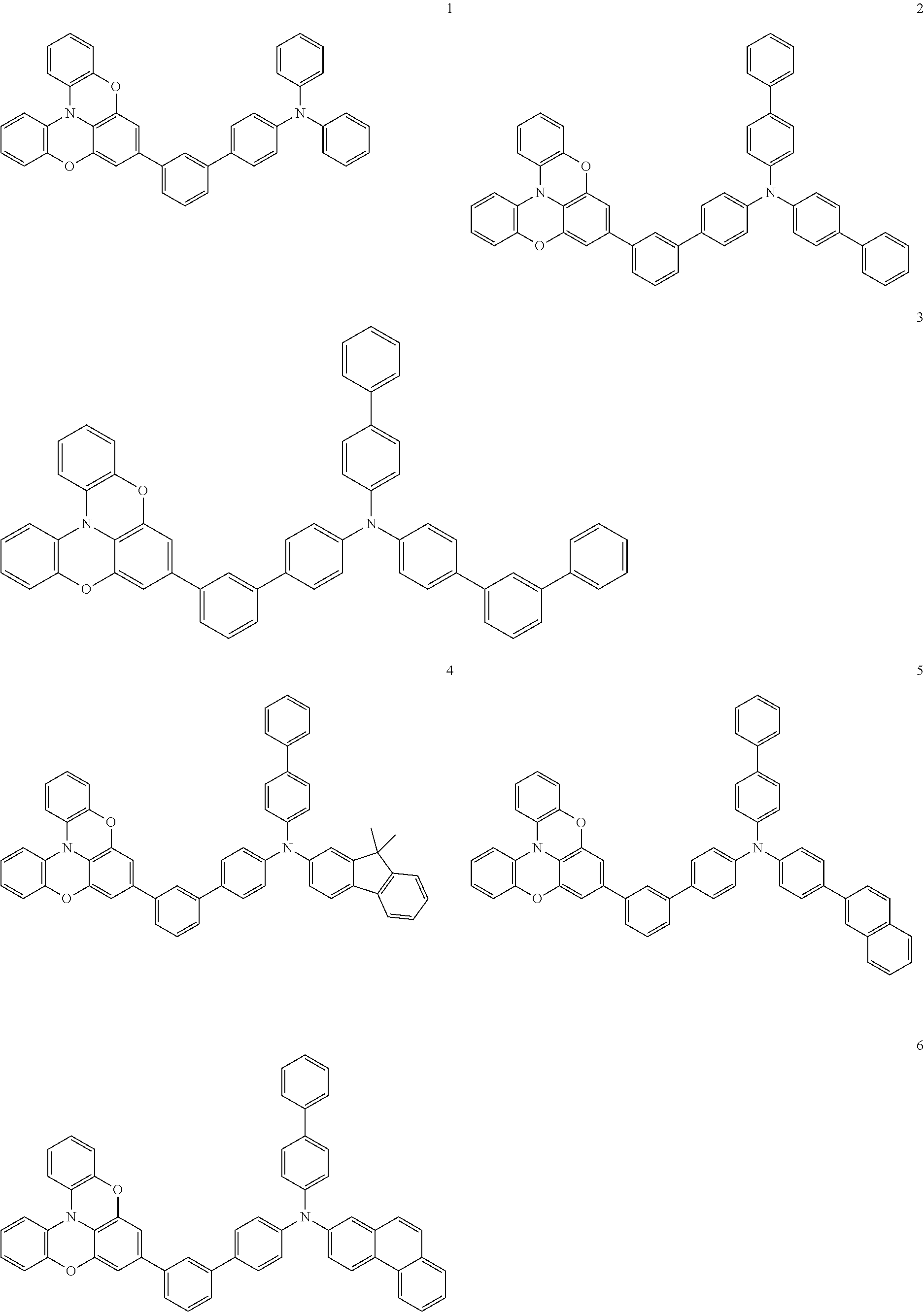

An example of the material for an organic EL device may be a compound represented by one of the following formulae 7 to 12.

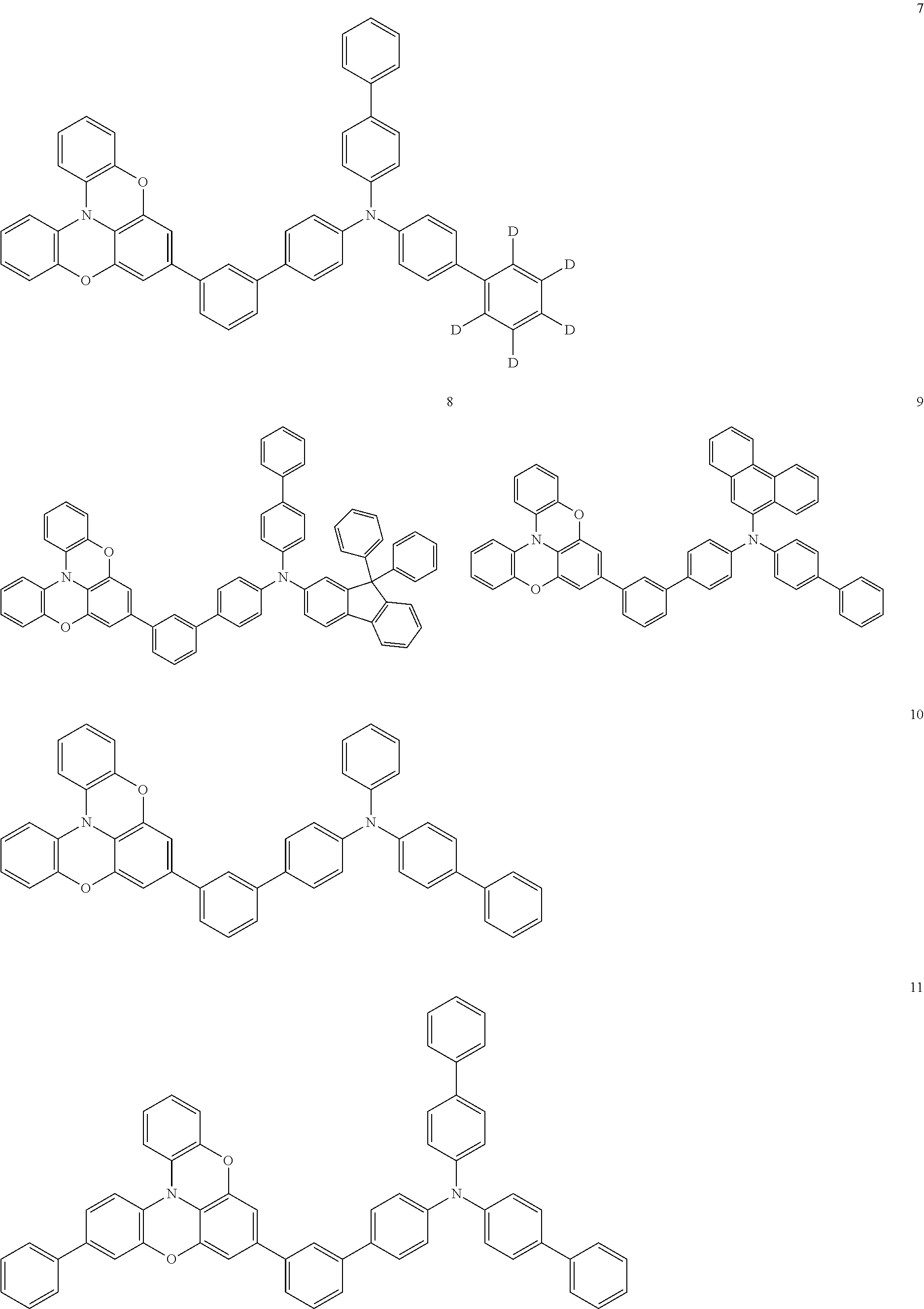

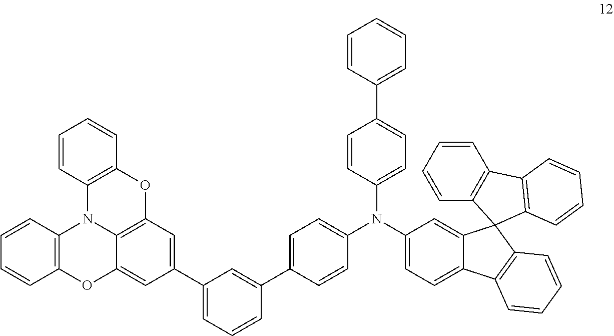 [SMILES: c1ccc(-c2ccc(N(c3ccc(-c4cccc(-c5cc6c7c(c5)Oc5ccccc5N7c5ccccc5O6)c4)cc3)c3ccc4c(c3)C3(c5ccccc5-c5ccccc53)c3ccccc3-4)cc2)cc1]

An example of the material for an organic EL device may be a compound represented by one of the following formulae 13 to 18.

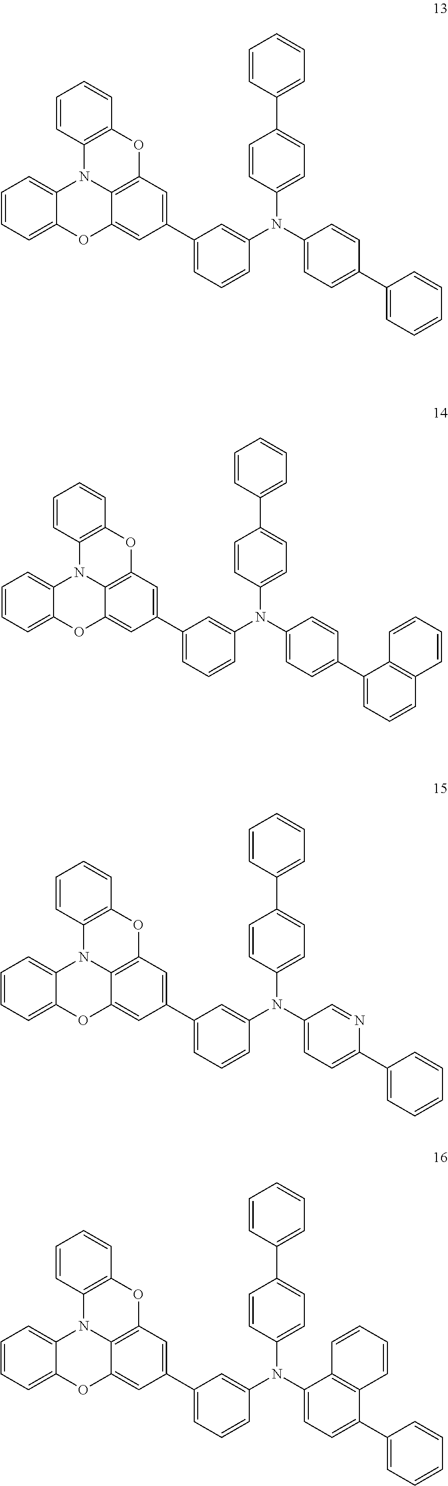 [SMILES: c1ccc(-c2ccc(N(c3ccc(-c4ccccc4)cc3)c3cccc(-c4cc5c6c(c4)Oc4ccccc4N6c4ccccc4O5)c3)cc2)cc1 ; c1ccc(-c2ccc(N(c3ccc(-c4cccc5ccccc45)cc3)c3cccc(-c4cc5c6c(c4)Oc4ccccc4N6c4ccccc4O5)c3)cc2)cc1 ; c1ccc(-c2ccc(N(c3ccc(-c4ccccc4)nc3)c3cccc(-c4cc5c6c(c4)Oc4ccccc4N6c4ccccc4O5)c3)cc2)cc1 ; c1ccc(-c2ccc(N(c3cccc(-c4cc5c6c(c4)Oc4ccccc4N6c4ccccc4O5)c3)c3ccc(-c4ccccc4)c4ccccc34)cc2)cc1]

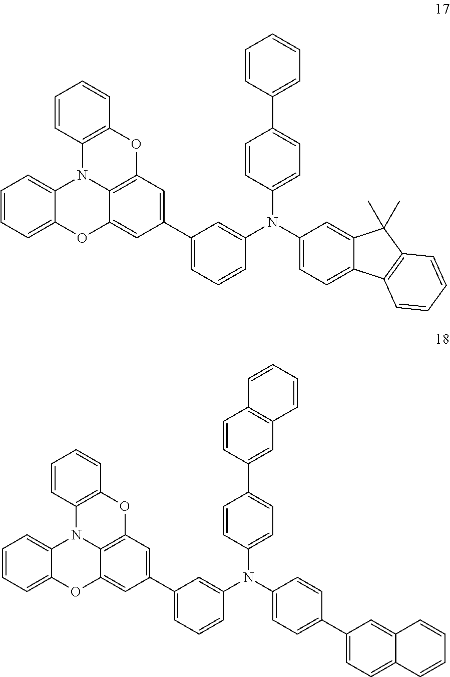 [SMILES: CC1(C)c2ccccc2-c2ccc(N(c3ccc(-c4ccccc4)cc3)c3cccc(-c4cc5c6c(c4)Oc4ccccc4N6c4ccccc4O5)c3)cc21 ; c1cc(-c2cc3c4c(c2)Oc2ccccc2N4c2ccccc2O3)cc(N(c2ccc(-c3ccc4ccccc4c3)cc2)c2ccc(-c3ccc4ccccc4c3)cc2)c1]

An example of the material for an organic EL device may be a compound represented by one of the following formulae 19 to 24.

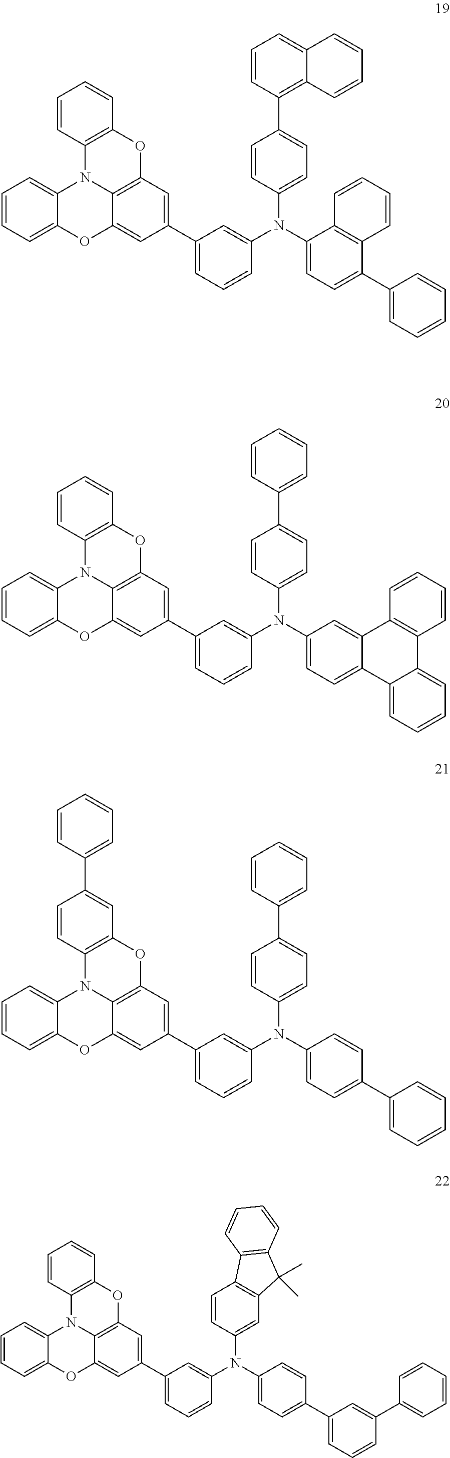 [SMILES: c1ccc(-c2ccc(N(c3ccc(-c4cccc5ccccc45)cc3)c3cccc(-c4cc5c6c(c4)Oc4ccccc4N6c4ccccc4O5)c3)c3ccccc23)cc1 ; c1ccc(-c2ccc(N(c3cccc(-c4cc5c6c(c4)Oc4ccccc4N6c4ccccc4O5)c3)c3ccc4c5ccccc5c5ccccc5c4c3)cc2)cc1 ; c1ccc(-c2ccc(N(c3ccc(-c4ccccc4)cc3)c3cccc(-c4cc5c6c(c4)Oc4cc(-c7ccccc7)ccc4N6c4ccccc4O5)c3)cc2)cc1 ; CC1(C)c2ccccc2-c2ccc(N(c3ccc(-c4cccc(-c5ccccc5)c4)cc3)c3cccc(-c4cc5c6c(c4)Oc4ccccc4N6c4ccccc4O5)c3)cc21]

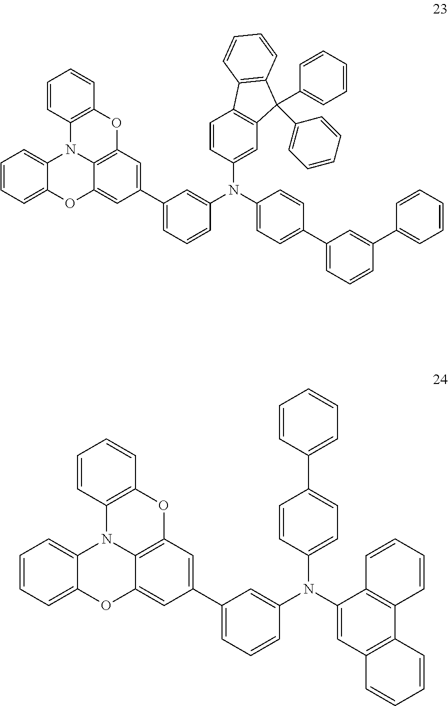 [SMILES: c1ccc(-c2cccc(-c3ccc(N(c4cccc(-c5cc6c7c(c5)Oc5ccccc5N7c5ccccc5O6)c4)c4ccc5c(c4)C(c4ccccc4)(c4ccccc4)c4ccccc4-5)cc3)c2)cc1 ; c1ccc(-c2ccc(N(c3cccc(-c4cc5c6c(c4)Oc4ccccc4N6c4ccccc4O5)c3)c3cc4ccccc4c4ccccc34)cc2)cc1]

An example of the material for an organic EL device may be a compound represented by one of the following formulae 25 to 30.

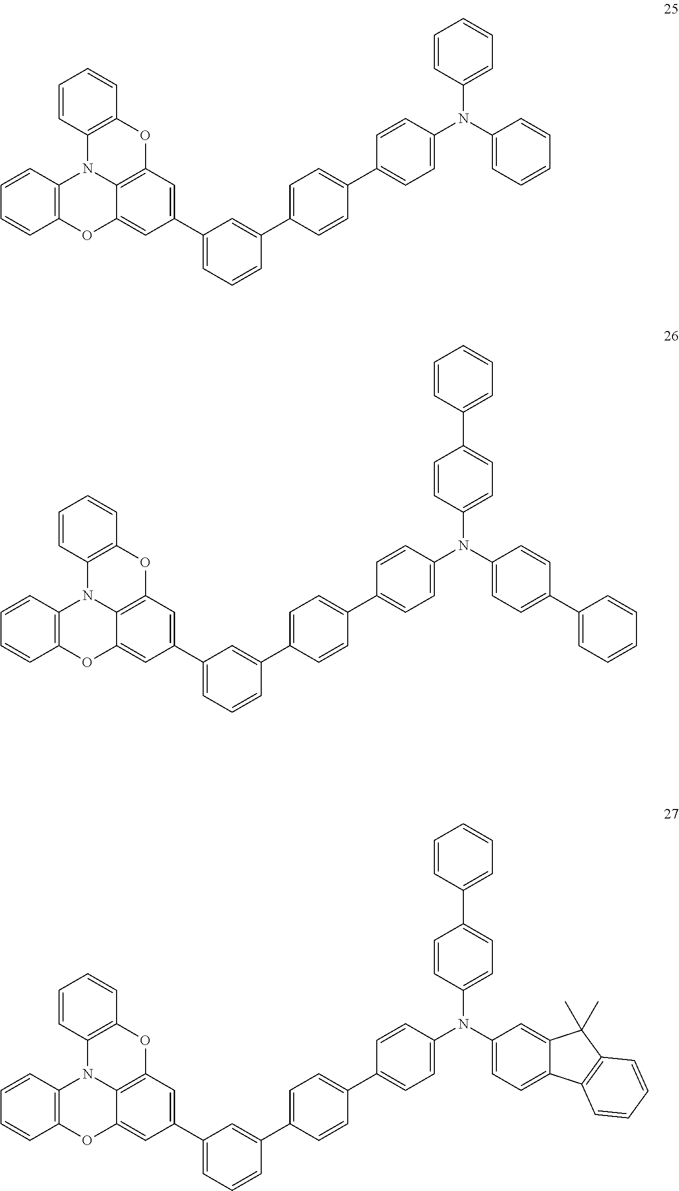 [SMILES: c1ccc(N(c2ccccc2)c2ccc(-c3ccc(-c4cccc(-c5cc6c7c(c5)Oc5ccccc5N7c5ccccc5O6)c4)cc3)cc2)cc1 ; c1ccc(-c2ccc(N(c3ccc(-c4ccccc4)cc3)c3ccc(-c4ccc(-c5cccc(-c6cc7c8c(c6)Oc6ccccc6N8c6ccccc6O7)c5)cc4)cc3)cc2)cc1 ; CC1(C)c2ccccc2-c2ccc(N(c3ccc(-c4ccccc4)cc3)c3ccc(-c4ccc(-c5cccc(-c6cc7c8c(c6)Oc6ccccc6N8c6ccccc6O7)c5)cc4)cc3)cc21]

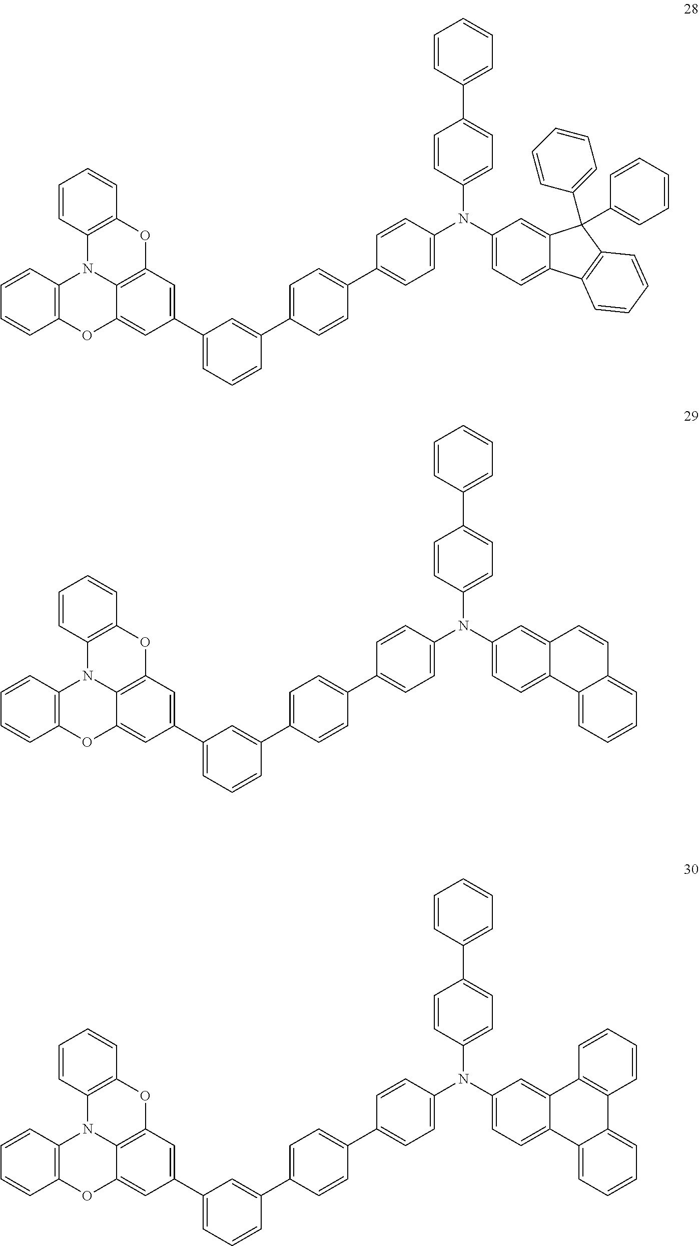 [SMILES: c1ccc(-c2ccc(N(c3ccc(-c4ccc(-c5cccc(-c6cc7c8c(c6)Oc6ccccc6N8c6ccccc6O7)c5)cc4)cc3)c3ccc4c(c3)C(c3ccccc3)(c3ccccc3)c3ccccc3-4)cc2)cc1 ; c1ccc(-c2ccc(N(c3ccc(-c4ccc(-c5cccc(-c6cc7c8c(c6)Oc6ccccc6N8c6ccccc6O7)c5)cc4)cc3)c3ccc4c(ccc5ccccc54)c3)cc2)cc1 ; c1ccc(-c2ccc(N(c3ccc(-c4ccc(-c5cccc(-c6cc7c8c(c6)Oc6ccccc6N8c6ccccc6O7)c5)cc4)cc3)c3ccc4c5ccccc5c5ccccc5c4c3)cc2)cc1]

An example of the material for an organic EL device may be a compound represented by one of the following formulae 31-36.

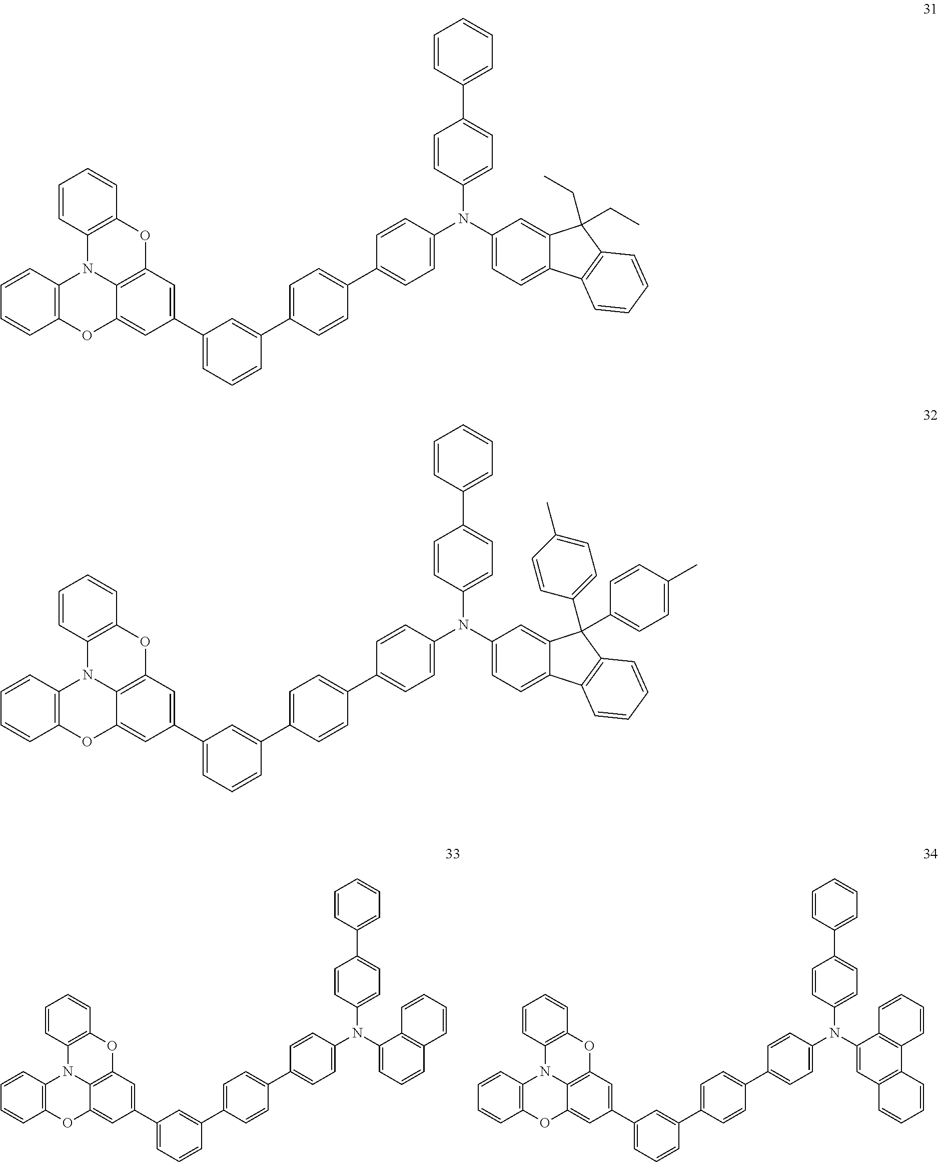 [SMILES: CCC1(CC)c2ccccc2-c2ccc(N(c3ccc(-c4ccccc4)cc3)c3ccc(-c4ccc(-c5cccc(-c6cc7c8c(c6)Oc6ccccc6N8c6ccccc6O7)c5)cc4)cc3)cc21 ; Cc1ccc(C2(c3ccc(C)cc3)c3ccccc3-c3ccc(N(c4ccc(-c5ccccc5)cc4)c4ccc(-c5ccc(-c6cccc(-c7cc8c9c(c7)Oc7ccccc7N9c7ccccc7O8)c6)cc5)cc4)cc32)cc1 ; c1ccc(-c2ccc(N(c3ccc(-c4ccc(-c5cccc(-c6cc7c8c(c6)Oc6ccccc6N8c6ccccc6O7)c5)cc4)cc3)c3cccc4ccccc34)cc2)cc1 ; c1ccc(-c2ccc(N(c3ccc(-c4ccc(-c5cccc(-c6cc7c8c(c6)Oc6ccccc6N8c6ccccc6O7)c5)cc4)cc3)c3cc4ccccc4c4ccccc34)cc2)cc1]

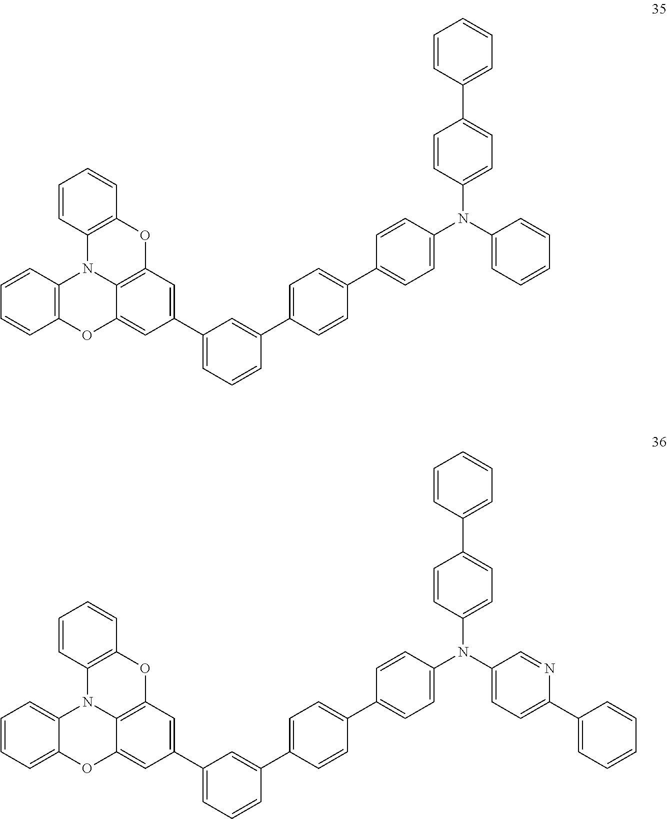 [SMILES: c1ccc(-c2ccc(N(c3ccccc3)c3ccc(-c4ccc(-c5cccc(-c6cc7c8c(c6)Oc6ccccc6N8c6ccccc6O7)c5)cc4)cc3)cc2)cc1 ; c1ccc(-c2ccc(N(c3ccc(-c4ccc(-c5cccc(-c6cc7c8c(c6)Oc6ccccc6N8c6ccccc6O7)c5)cc4)cc3)c3ccc(-c4ccccc4)nc3)cc2)cc1]

An example of the material for an organic EL device may be a compound represented by one of the following formulae 37 to 42.

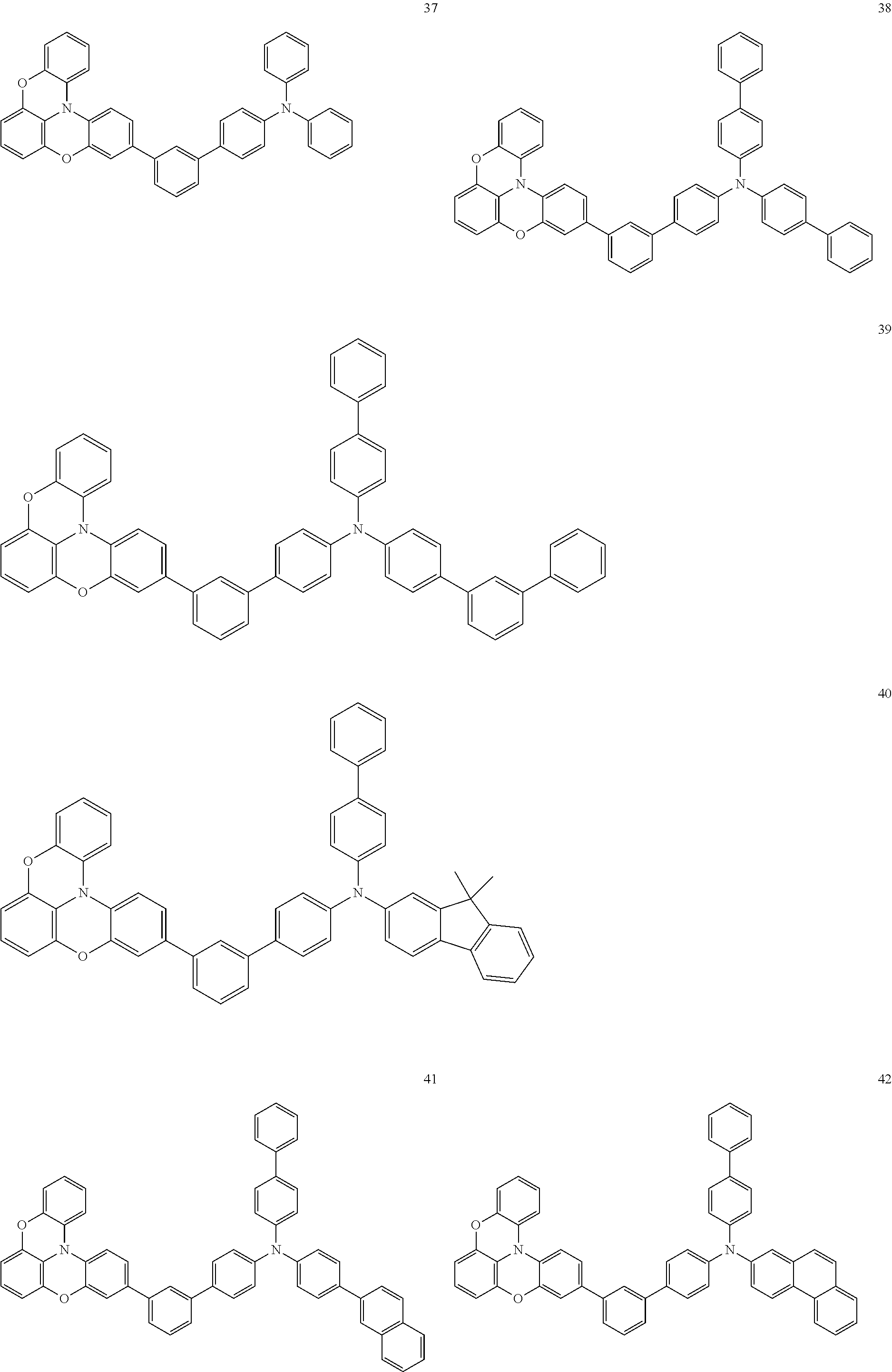

An example of the material for an organic EL device may be a compound represented by one of the following formulae 43-48.

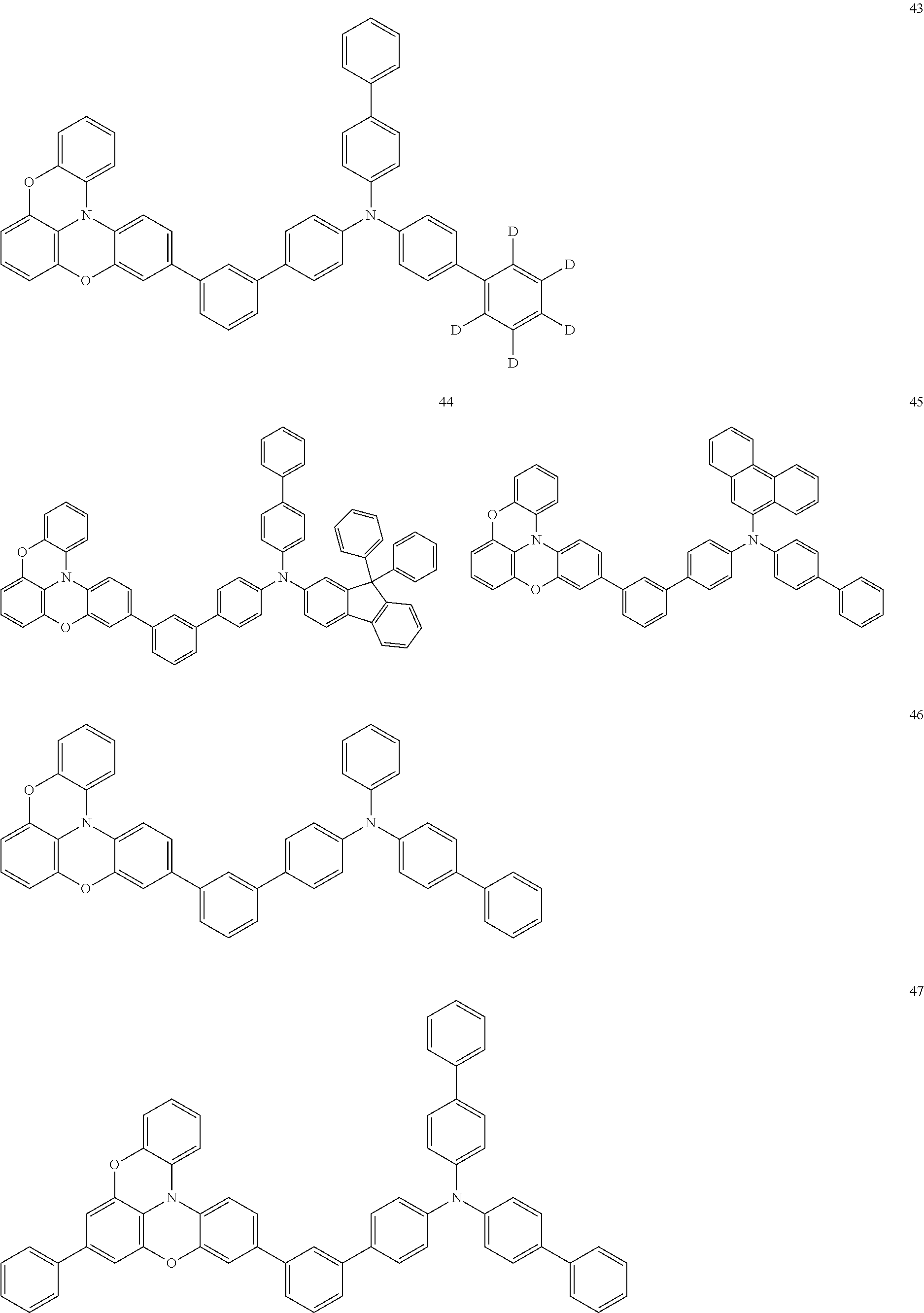

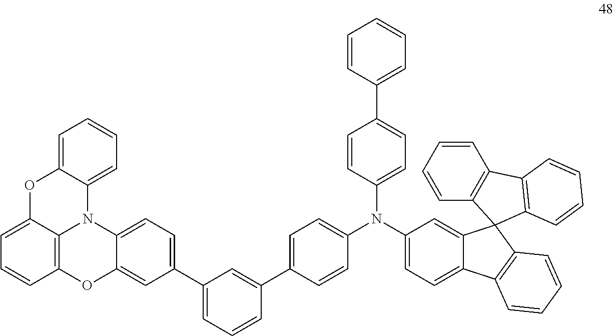 [SMILES: c1ccc(-c2ccc(N(c3ccc(-c4cccc(-c5ccc6c(c5)Oc5cccc7c5N6c5ccccc5O7)c4)cc3)c3ccc4c(c3)C3(c5ccccc5-c5ccccc53)c3ccccc3-4)cc2)cc1]

An example of the material for an organic EL device may be a compound represented by one of the following formulae 49 to 54.

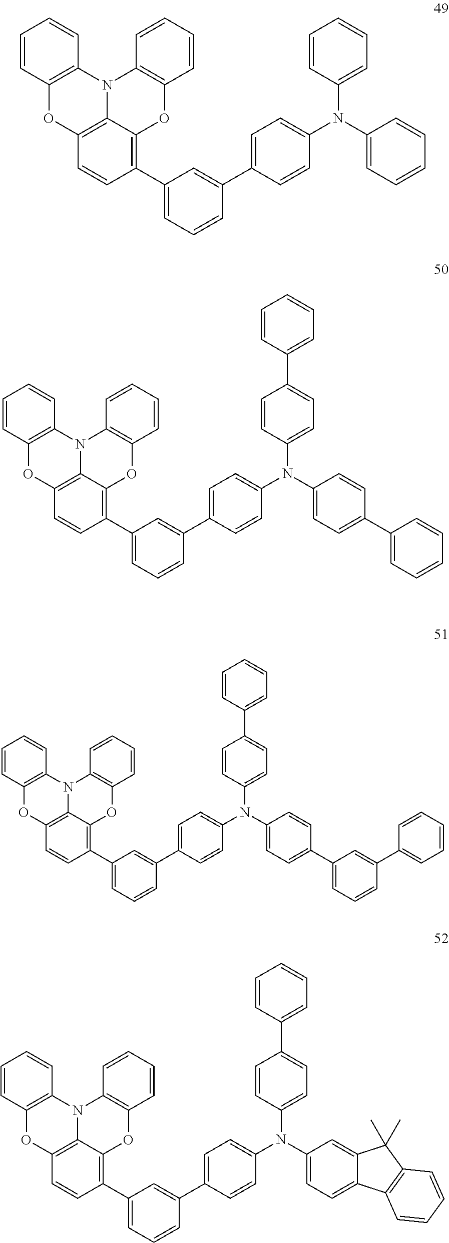 [SMILES: c1ccc(N(c2ccccc2)c2ccc(-c3cccc(-c4ccc5c6c4Oc4ccccc4N6c4ccccc4O5)c3)cc2)cc1 ; c1ccc(-c2ccc(N(c3ccc(-c4ccccc4)cc3)c3ccc(-c4cccc(-c5ccc6c7c5Oc5ccccc5N7c5ccccc5O6)c4)cc3)cc2)cc1 ; c1ccc(-c2ccc(N(c3ccc(-c4cccc(-c5ccccc5)c4)cc3)c3ccc(-c4cccc(-c5ccc6c7c5Oc5ccccc5N7c5ccccc5O6)c4)cc3)cc2)cc1 ; CC1(C)c2ccccc2-c2ccc(N(c3ccc(-c4ccccc4)cc3)c3ccc(-c4cccc(-c5ccc6c7c5Oc5ccccc5N7c5ccccc5O6)c4)cc3)cc21]

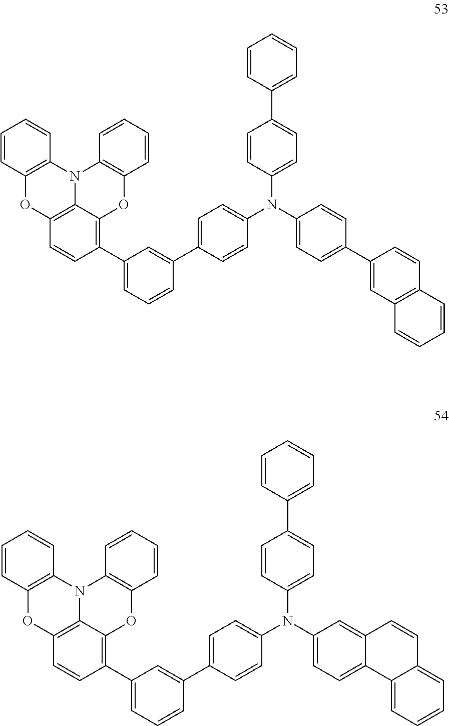 [SMILES: c1ccc(-c2ccc(N(c3ccc(-c4cccc(-c5ccc6c7c5Oc5ccccc5N7c5ccccc5O6)c4)cc3)c3ccc(-c4ccc5ccccc5c4)cc3)cc2)cc1 ; c1ccc(-c2ccc(N(c3ccc(-c4cccc(-c5ccc6c7c5Oc5ccccc5N7c5ccccc5O6)c4)cc3)c3ccc4c(ccc5ccccc54)c3)cc2)cc1]

An example of the material for an organic EL device may be a compound represented by one of the following formulae 55 to 60.

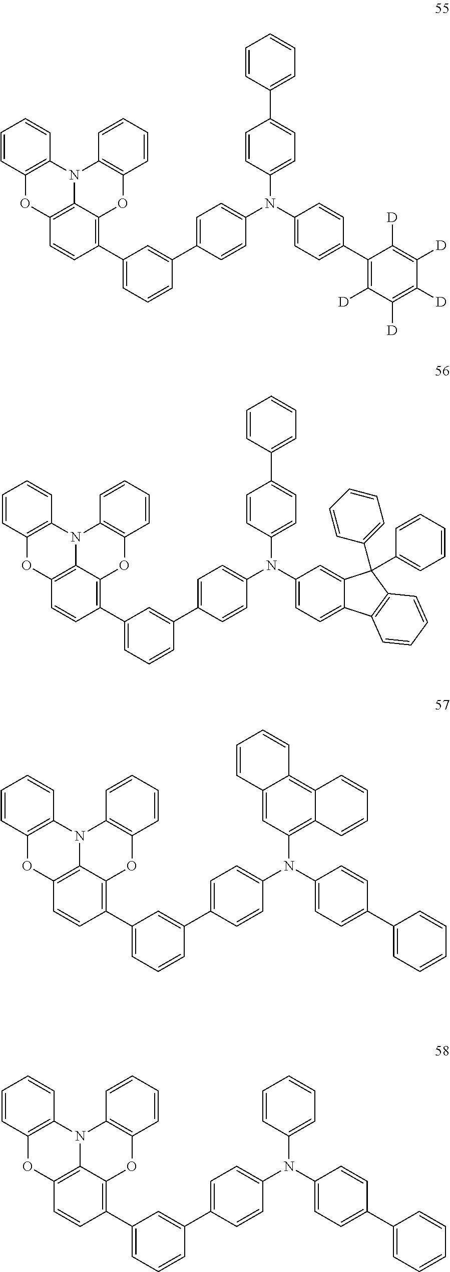 [SMILES: [2H]c1c([2H])c([2H])c(-c2ccc(N(c3ccc(-c4ccccc4)cc3)c3ccc(-c4cccc(-c5ccc6c7c5Oc5ccccc5N7c5ccccc5O6)c4)cc3)cc2)c([2H])c1[2H] ; c1ccc(-c2ccc(N(c3ccc(-c4cccc(-c5ccc6c7c5Oc5ccccc5N7c5ccccc5O6)c4)cc3)c3ccc4c(c3)C(c3ccccc3)(c3ccccc3)c3ccccc3-4)cc2)cc1 ; c1ccc(-c2ccc(N(c3ccc(-c4cccc(-c5ccc6c7c5Oc5ccccc5N7c5ccccc5O6)c4)cc3)c3cc4ccccc4c4ccccc34)cc2)cc1 ; c1ccc(-c2ccc(N(c3ccccc3)c3ccc(-c4cccc(-c5ccc6c7c5Oc5ccccc5N7c5ccccc5O6)c4)cc3)cc2)cc1]

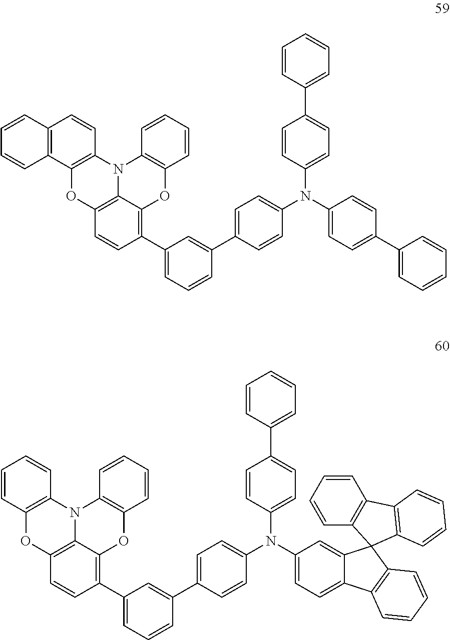 [SMILES: c1ccc(-c2ccc(N(c3ccc(-c4ccccc4)cc3)c3ccc(-c4cccc(-c5ccc6c7c5Oc5ccccc5N7c5ccc7ccccc7c5O6)c4)cc3)cc2)cc1 ; c1ccc(-c2ccc(N(c3ccc(-c4cccc(-c5ccc6c7c5Oc5ccccc5N7c5ccccc5O6)c4)cc3)c3ccc4c(c3)C3(c5ccccc5-c5ccccc53)c3ccccc3-4)cc2)cc1]

The material for an organic EL device may be used in one layer of stacking layers disposed between the emission layer and the anode of an organic device. The amorphous property may be maintained, the high mobility of holes may be realized, and the decrease of the driving voltage and the high emission efficiency of the organic EL device may be realized.

(Organic EL Device)

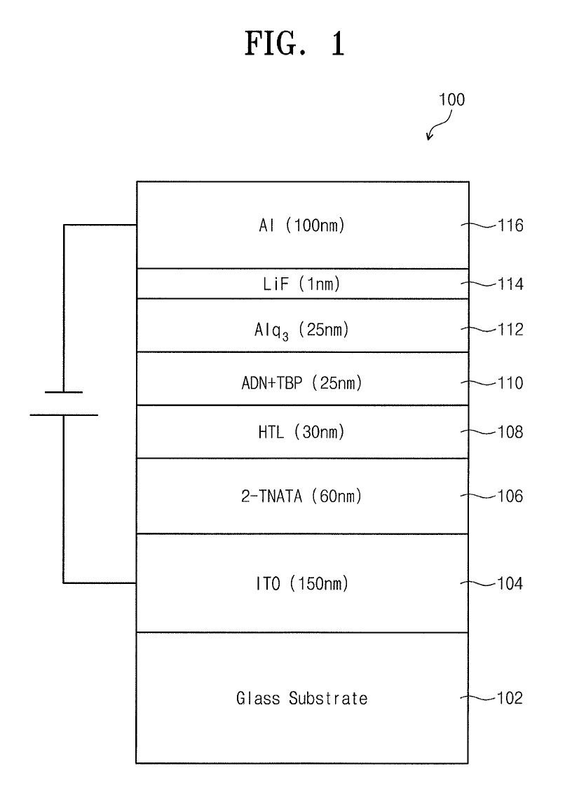

An organic EL device using the material for an organic EL device will be explained. FIG. 1 is a schematic diagram showing an organic EL device 100 according to an embodiment. The organic EL device 100 may include, e.g. a substrate 102, an anode 104, a hole injection layer 106, a hole transport layer 108, an emission layer 110, an electron transport layer 112, an electron injection layer 114 and a cathode 116. In FIG. 1, specific materials and thicknesses are illustrated as an example, as described more fully below. In an embodiment, the material for an organic EL device may be used in one layer of stacking layers disposed between the emission layer and the anode of the organic EL device.

For example, a case of using the material for an organic EL device in a hole transport layer 108 will be explained. The substrate 102 may be, e.g. a transparent glass substrate, a semiconductor substrate using silicon, etc., or a flexible substrate of a resin, etc. The anode 104 may be disposed on the substrate 102. The anode 104 may be formed using indium tin oxide (ITO), indium zinc oxide (ILO), etc. The hole injection layer 106 may be disposed on the anode 104. The hole injection layer 106 may include, e.g. 4,4′,4″-tris[2-naphthyl(phenyl)amino]triphenylamine (2-TNATA), N,N,N′,N′-tetrakis(3-methylphenyl)-3,3′-dimethylbenzidine (HMTPD), etc. The hole transport layer 108 may be disposed on the hole injection layer 106. The hole transport layer may be formed using the material for an organic EL device according to embodiments. In an implementation, the thickness of the hole transport layer 108 may be from about 3 nm to about 100 nm.

The emission layer 110 may be disposed on the hole transport layer 108. The emission layer 110 may include a condensed polycyclic aromatic derivative selected from, for example, an anthracene derivative, a pyrene derivative, a fluoranthene derivative, a chrysene derivative, a benzoanthracene derivative and a triphenylene derivative. For example, the emission layer 110 may include an anthracene derivative or a pyrene derivative. As the anthracene derivative used in the emission layer 110, a compound represented by the following General Formula (5) may be used.

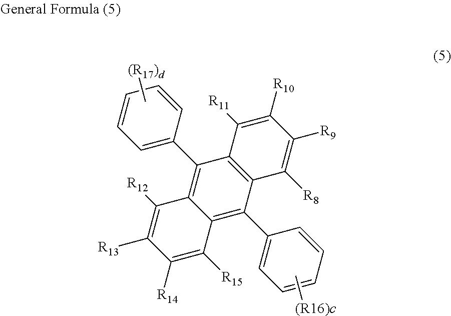

In General Formula (5), R₈, R₉, R₁₀, R₁₁, R₁₂, R₁₃, R₁₄, R₁₅, R₁₆ and R₁₇ are a substituted or unsubstituted aryl group having 6 to 30 ring carbon atoms, a substituted or unsubstituted heteroaryl group having 1 to 30 ring carbon atoms, an alkyl group having 1 to 15 carbon atoms, a silyl group, a halogen atom, a hydrogen atom or a deuterium atom. In addition, c and d are independently an integer from 0 to 5. Multiple adjacent R₈, R₉, R₁₀, R₁₁, R₁₂, R₁₃, R₁₄, R₁₅, R₁₆ and R₁₇ may combine and form a saturated or unsaturated ring.

The substituted or unsubstituted heteroaryl group having 1 to 30 ring carbon atoms used in R₈, R₉, R₁₀, R₁₁, R₁₂, R₁₃, R₁₄, R₁₅, R₁₆ and R₁₇ may independently include a benzothiazolyl group, a thiophenyl group, a thienothiophenyl group, a thienothienothiophenyl group, a benzothiophenyl group, a benzofuryl group, a dibenzothiophenyl group, a dibenzofuryl group, a N-arylcarbazolyl group, a N-heteroarylcarbazolyl group, a N-alkylcarbazolyl group, a phenoxazyl group, a phenothiazyl group, a pyridyl group, a pyrimidyl group, a triazole group, a quinolinyl group, a quinoxalyl group, etc., as examples

The alkyl group having 1 to 15 carbon atoms used in R₈, R₉, R₁₀, R₁₁, R₁₂, R₁₃, R₁₄, R₁₅, R₁₆ and R₁₇ may include a methyl group, an ethyl group, a propyl group, an isopropyl group, a n-butyl group, a s-butyl group, an isobutyl group, a t-butyl group, a n-pentyl group, a n-hexyl group, a n-heptyl group, a n-octyl group, a hydroxymethyl group, a 1-hydroxyethyl group, a 2-hydroxyethyl group, a 2-hydroxyisobutyl group, a 1,2-dihydroxyethyl group, a 1,3-dihydroxyisopropyl group, a 2,3-dihydroxy-t-butyl group, a 1,2,3-trihydroxypropyl group, a chloromethyl group, a 1-chloroethyl group, a 2-chloroethyl group, a 2-chloroisobutyl group, a 1,2-dichloroethyl group, a 1,3-dichloroisopropyl group, a 2,3-dichloro-t-butyl group, a 1,2,3-trichloropropyl group, a bromomethyl group, a 1-bromoethyl group, a 2-bromoethyl group, a 2-bromoisobutyl group, a 1,2-dibromoethyl group, a 1,3-dibromoisopropyl group, a 2,3-dibromo-t-butyl group, a 1,2,3-tribromopropyl group, an iodomethyl group, a 1-iodoethyl group, a 2-iodoethyl group, a 2-iodoisobutyl group, a 1,2-diiodoethyl group, a 1,3-diiodoisopropyl group, a 2,3-diiodo-t-butyl group, a 1,2,3-triiodopropyl group, an aminomethyl group, a 1-aminoethyl group, a 2-aminoethyl group, a 2-aminoisobutyl group, a 1,2-diaminoethyl group, a 1,3-diaminoisopropyl group, a 2,3-diamino-t-butyl group, a 1,2,3-triaminopropyl group, a cyanomethyl group, a 1-cyanoethyl group, a 2-cyanoethyl group, a 2-cyanoisobutyl group, a 1,2-dicyanoethyl group, a 1,3-dicyanoisopropyl group, a 2,3-dicyano-t-butyl group, a 1,2,3-tricyanopropyl group, a nitromethyl group, a 1-nitroethyl group, a 2-nitroethyl group, a 2-nitroisobutyl group, a 1,2-dinitroethyl group, a 1,3-dinitroisopropyl group, a 2,3-dinitro-t-butyl group, a 1,2,3-trinitropropyl group, a cyclopropyl group, a cyclobutyl group, a cyclopentyl group, a cyclohexyl group, a 4-methylcyclohexyl group, a 1-adamantyl group, a 2-adamantyl group, a 1-norbornyl group, a 2-norbornyl group, etc, as examples.

The anthracene derivative used in the emission layer 110 of the organic EL device may be a compound represented by the following formulae a-1 to a-6.

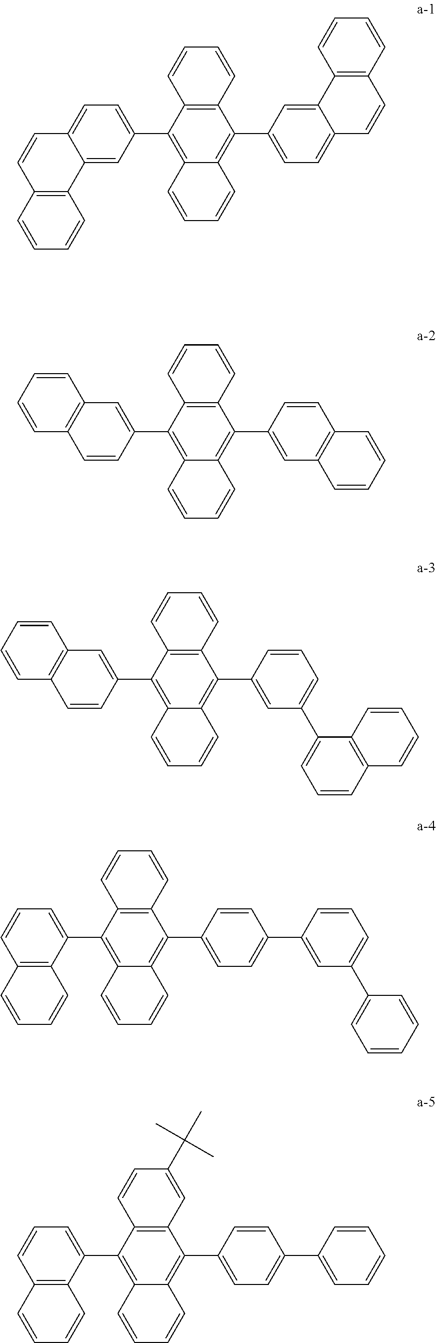

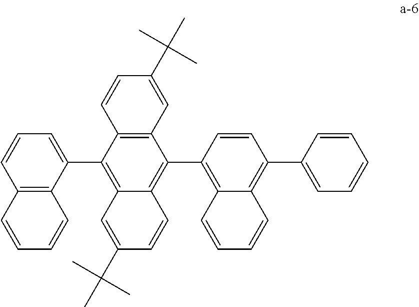 [SMILES: CC(C)(C)c1ccc2c(-c3ccc(-c4ccccc4)c4ccccc34)c3cc(C(C)(C)C)ccc3c(-c3cccc4ccccc34)c2c1]

The anthracene derivative used in the emission layer 110 of the organic EL device may be a compound represented by the following formulae a-7 to a-12.

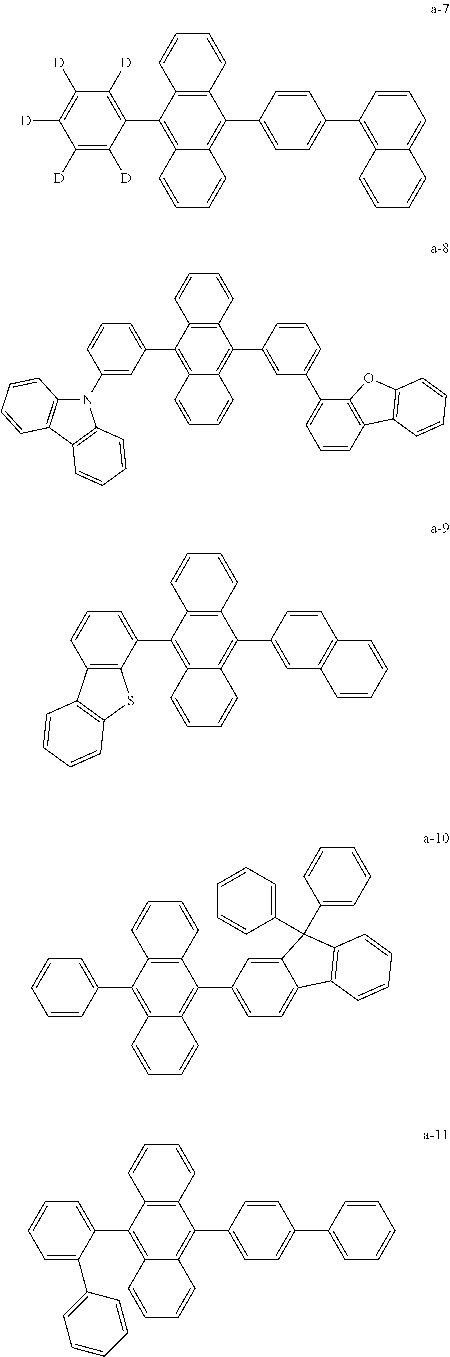

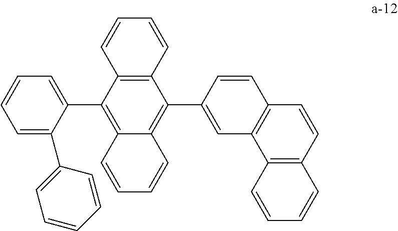 [SMILES: c1ccc(-c2ccccc2-c2c3ccccc3c(-c3ccc4ccc5ccccc5c4c3)c3ccccc23)cc1]

The emission layer 110 may include a styryl derivative (e.g. 1,4-bis[2-(3-N-ethylcarbazolyl)vinyl]benzene (BCzVB), 4-(di-p-tolylamino)-4′-[(di-p-tolylamino)styryl]stilbene (DPAVB), N-(4-((E)-2-(6-((E)-4-(diphenylamino)styryl)naphthalen-2-yl)vinyl)phenyl)-N-phenylbenzenamine (N-BDAVBi), perylene or a derivative thereof (e.g. 2,5,8,11-tetra-t-butylperylene (TBPe)), pyrene or a derivative thereof (e.g. 1,1-dipyrene, 1,4-dipyrenylbenzene, 1,4-bis(N,N-diphenylamino)pyrene), etc. doped with 2,5,8,11-tetra-t-butylperylene (TBP), etc., as examples. In some implementations, the emission layer 110 may include 9,10-di(naphthalene-2-yl)anthracene (ADN) doped with TBP.

The electron transport layer 112 may be disposed on the emission layer 110. The electron transport layer 112 may be formed using, a material including e.g. tris(8-hydroxyquinolinato)aluminum (Alq3) or a material having a nitrogen-containing aromatic ring (e.g., a material including a pyridine ring such as 1,3,5-tri[(3-pyridyl)-phen-3-yl]benzene, a material including a triazine ring such as 2,4,6-tris(3′-(pyridin-3-yl)biphenyl-3-yl)-1,3,5-triazine, or a material including an imidazole derivative such as 2-(4-(N-phenylbenzoimidazolyl-1-ylphenyl)-9,10-dinaphthylanthracene).

The electron injection layer 114 may be disposed on the electron transport layer 112. The electron injection layer 114 may be formed using a material including, e.g. lithium fluoride (LiF), lithium-8-quinolinato (Liq), etc. The cathode 116 may be disposed on the electron injection layer 114. The cathode 116 may be formed using a metal such as aluminum (Al), silver (Ag), lithium (Li), magnesium (Mg) and calcium (Ca), or a transparent material such as ITO, IZO, etc. The thin layers may be formed by selecting an appropriate layer forming method according to materials such as a vacuum evaporation method, a sputtering method, diverse coating methods, etc.

In the organic EL device 100 according to an embodiment, a hole transport layer providing the decrease of the driving voltage and the high efficiency may be realized by using the material for an organic EL device. In addition, the material for an organic EL device may be applied in an organic EL display of an active matrix type using a TFT.

In addition, in the organic EL device 100 according to an embodiment, the decrease of the driving voltage and the high emission efficiency of the organic EL device may be realized by using the material for an organic EL device in one layer of stacking layers disposed between the emission layer and the anode.

The following Examples and Comparative Examples are provided in order to highlight characteristics of one or more embodiments, but it will be understood that the Examples and Comparative Examples are not to be construed as limiting the scope of the embodiments, nor are the Comparative Examples to be construed as being outside the scope of the embodiments. Further, it will be understood that the embodiments are not limited to the particular details described in the Examples and Comparative Examples.

### Examples

### Preparation Method

The above-mentioned material for an organic EL device may be synthesized, for example, as follows. For example, Compound 2 may be synthesized by the following reactions. First, Compound A was synthesized as an intermediate as follows.

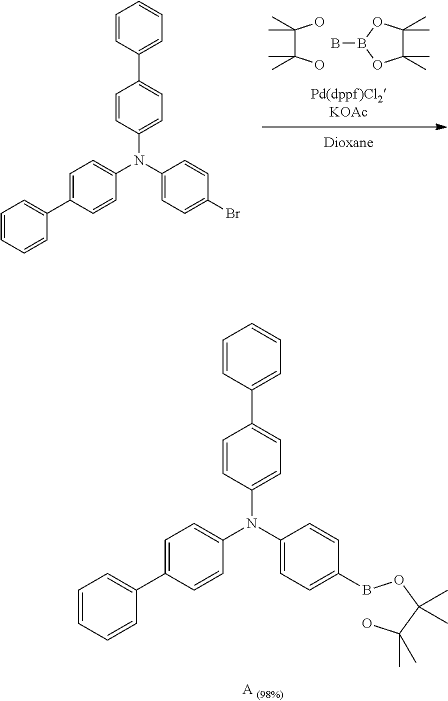

Under an argon atmosphere, 53.8 g of N-[1,1′-biphenyl]-4-yl-N-(4-bromophenyl)-[1,1′-biphenyl]-4-amine, 6.46 g of Pd(dppf)Cl₂.CH₂Cl₂, 33.3 g of KOAc, and 33.0 g of bis(pinacolato)diboron were added to a 2 L flask, followed by degassing in 750 mL of a dioxane solvent and stirring at about 100° C. for about 12 hours. Then, the solvent was distilled, CH₂Cl₂ and water were added, an organic phase was separated, magnesium sulfate and activated carbon were added, suction filtration was performed, and solvents were distilled. The crude product thus obtained was separated using a silica gel column chromatography (using a mixture solvent of dichloromethane and hexane) to produce 56.8 g of a target material of a white solid (yield 98%). The molecular weight of Compound A measured by FAB-MS measurement was 523.

By using Compound A as a raw material, Compound B may be synthesized by, for example, the following reaction.

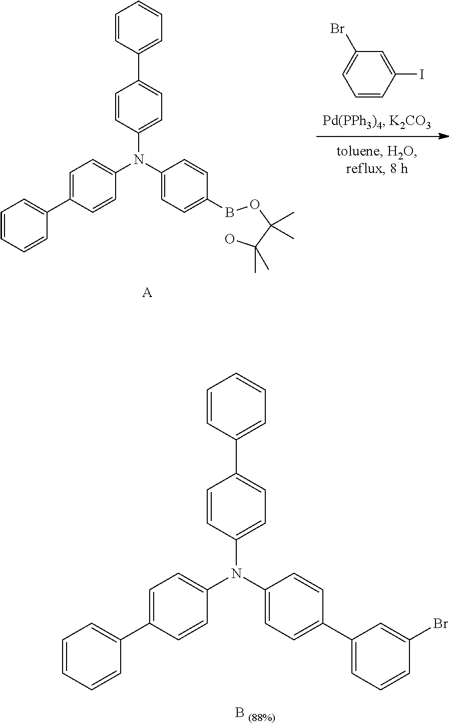

Under an argon atmosphere, 10.0 g of Compound A, 6.00 g of 1-iodo-3-bromobenzene, 1.54 g of Pd(PPh₃)₄, and 5.25 g of potassium carbonate were added to a 300 mL, three-necked flask, followed by heating and stirring in a mixture solvent of 450 mL of toluene and 60 mL of water at about 90° C. for about 8 hours. After cooling in the air, water was added to separate an organic layer, and solvents were distilled. The crude product thus obtained was separated using a silica gel column chromatography (using a mixture solvent of dichloromethane and hexane) and recrystallized in a mixture solvent of toluene and hexane to obtain 9.29 g of Compound B of a white solid (yield 88%). The molecular weight of Compound B measured by FAB-MS measurement was 553.

In addition, Compound C may be synthesized by, for example, the following reaction.

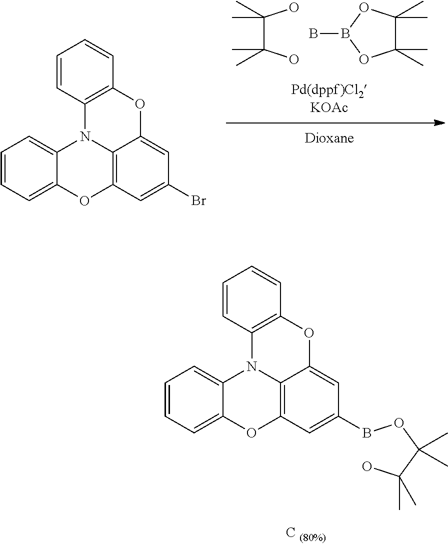

Under an argon atmosphere, 3.00 g of 7-bromo-[1,4]benzoxazino[2,3,4-kl]phenoxazine, 0.48 g of Pd(dppf)Cl₂.CH₂Cl₂, 2.51 g of KOAc and 2.81 g of bis(pinacolato)diboron were added to a 200 mL flask, followed by degassing in 75 mL of a dioxane solvent and stirring at about 100° C. for about 12 hours. Then, the solvent was distilled, CH₂Cl₂ and water were added, an organic phase was separated, magnesium sulfate and activated carbon were added, suction filtration was performed, and solvents were distilled. The crude product thus obtained was separated using a silica gel column chromatography (using a mixture solvent of dichloromethane and hexane) to produce 2.72 g of target material C of a yellow solid (yield 80%). The molecular weight of Compound C measured by FAB-MS measurement was 399.

By using Compounds B and C as raw materials, Compound 2 may be synthesized by, for example, the following reaction.

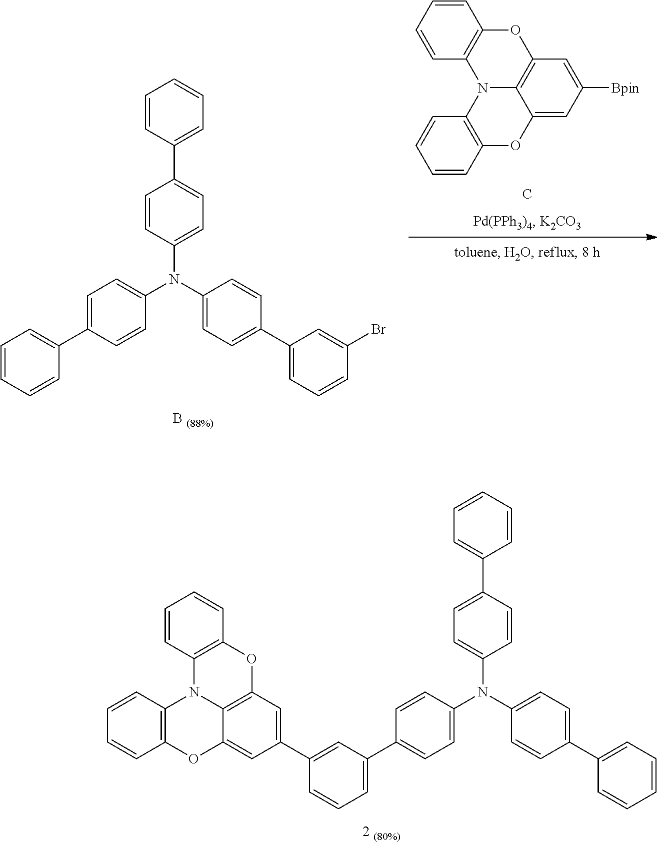

Under an argon atmosphere, 3.70 g of Compound B, 5.08 g of Compound C, 0.94 g of Pd(PPh₃)₄, and 2.56 g of potassium carbonate were added to a 200 mL, three-necked flask, followed by heating and stirring in a mixture solvent of 70 mL of toluene and 20 mL of water at about 90° C. for about 8 hours. After cooling in the air, water was added to separate an organic layer, and solvents were distilled. The crude product thus obtained was dissolved in hot toluene and activated carbon was added, followed by stirring. Then, filtration and distillation under a reduced pressure were performed to obtain 5.10 g of Compound 2 of a yellow solid (yield 86%).

The molecular weight of Compound 2 measured by FAB-MS measurement was 745. The chemical shift values (6) of Compound 2 measured by ¹H-NMR (CDCl₃) were 7.70 (s, 1H), 7.57-7.50 (m, 15H), 7.48-7.41 (m, 4H), 6.91-6.60 (m, 10H).

Compounds 10, 13, and 38 were synthesized by a similar method as Compound 2 by using a different bromophenylamine for Compound B.

Organic EL devices of Examples 1, 2, 3 and 4 were manufactured using the above-mentioned Compounds 2, 10, 13 and 38 as the hole transport materials by the above-mentioned preparation method.

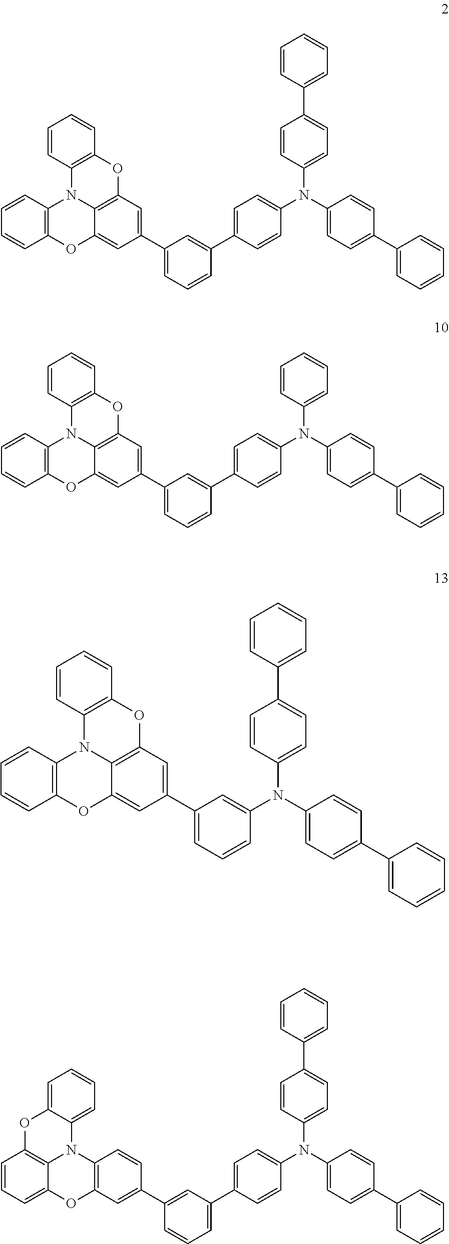 [SMILES: c1ccc(-c2ccc(N(c3ccc(-c4ccccc4)cc3)c3ccc(-c4cccc(-c5cc6c7c(c5)Oc5ccccc5N7c5ccccc5O6)c4)cc3)cc2)cc1 ; c1ccc(-c2ccc(N(c3ccccc3)c3ccc(-c4cccc(-c5cc6c7c(c5)Oc5ccccc5N7c5ccccc5O6)c4)cc3)cc2)cc1 ; c1ccc(-c2ccc(N(c3ccc(-c4ccccc4)cc3)c3cccc(-c4cc5c6c(c4)Oc4ccccc4N6c4ccccc4O5)c3)cc2)cc1 ; c1ccc(-c2ccc(N(c3ccc(-c4ccccc4)cc3)c3ccc(-c4cccc(-c5ccc6c(c5)Oc5cccc7c5N6c5ccccc5O7)c4)cc3)cc2)cc1]

In addition, organic EL devices of Comparative Examples 1, 2 and 3 were manufactured using the following Compounds 61, 62 and 63 as the hole transport materials for comparison.

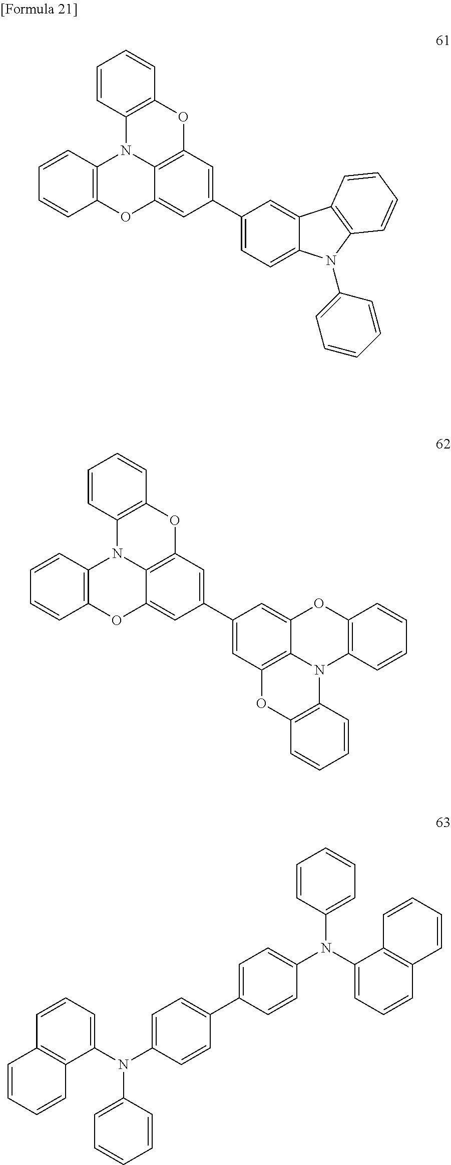 [SMILES: c1ccc(-n2c3ccccc3c3cc(-c4cc5c6c(c4)Oc4ccccc4N6c4ccccc4O5)ccc32)cc1 ; c1ccc2c(c1)Oc1cc(-c3cc4c5c(c3)Oc3ccccc3N5c3ccccc3O4)cc3c1N2c1ccccc1O3 ; c1ccc(N(c2ccc(-c3ccc(N(c4ccccc4)c4cccc5ccccc45)cc3)cc2)c2cccc3ccccc23)cc1]

In an embodiment, a transparent glass substrate was used as the substrate 102, the anode 104 was formed using ITO to a layer thickness of about 150 nm, the hole injection layer 106 was formed using 2-TNATA to a layer thickness of about 60 nm, the hole transport layer 108 was formed using the compounds of examples and comparative examples to a layer thickness of about 70 nm, the emission layer 110 was formed using ADN doped with 3% TBP to a layer thickness of about 25 nm, the electron transport layer 112 was formed using Alq3 to a layer thickness of about 25 nm, the electron injection layer 114 was formed using LiF to a layer thickness of about 1 nm, and the cathode 116 was formed using Al to a layer thickness of about 100 nm.

For the organic EL devices thus manufactured, voltage and emission efficiency were evaluated. In addition, the evaluation was performed with current density of about 20 mA/cm².

TABLE 1

| Example of | | Current | | Emission |
|---|---|---|---|---|
| manufacturing | Hole transport | density | Voltage | efficiency |
| device | material | (mA/cm²) | (V) | (cd/A) |
| Example 1 | Compound 2 | 10 | 6.6 | 6.7 |
| Example 2 | Compound 10 | 10 | 6.6 | 6.7 |
| Example 3 | Compound 13 | 10 | 6.7 | 6.4 |
| Example 4 | Compound 38 | 10 | 6.8 | 6.0 |
| Comparative | Compound 61 | 10 | 7.3 | 5.3 |
| Example 1 | | | | |
| Comparative | Compound 62 | 10 | 7.2 | 5.2 |
| Example 2 | | | | |
| Comparative | Compound 63 | 10 | 7.4 | 4.9 |
| Example 3 | | | | |

From the results in Table 1, it can be seen that when the materials for organic EL devices of Examples 1, 2, 3 and 4 introducing a benzoxazinophenoxazine part having high planarity to the meta position of a phenylene group with small conjugation effect in an amine having high amorphous property and high hole mobility are applied to the hole transport layers of the organic EL devices, driving at a low voltage and high emission efficiency may be obtained when compared to those of the organic EL devices obtained by using the compounds of the comparative examples. Thus, it can be seen that the amorphous property may be maintained, and the high mobility of holes may be realized by using the materials for organic EL devices according to Examples 1, 2, 3 and 4 obtained by introducing the benzoxazinophenoxazine part with high planarity to the meta position of a phenylene group with small conjugation effect in an amine having high amorphous property and high hole mobility.

In addition, in the organic EL devices of Examples 1, 2 and 3 using the compounds introducing a benzoxazinophenoxazine part represented by Formula (2) to General Formula (1), a driving voltage may be decreased, and emission efficiency may be increased further when compared to the organic EL device of Example 4 introducing a benzoxazinophenoxazine part represented by Formula (3).

By way of summation and review, in the application of an organic EL device in a display, driving at a low voltage and high emission efficiency of the organic EL device are desirable. In a blue emission region, the driving voltage of the organic EL device may be high, and the emission efficiency thereof may be insufficient when compared to a red emission region and a green emission region. To realize the driving at a low voltage and high emission efficiency of the organic EL device, the normalization and stabilization of a hole transport layer may be applied. As the material of the organic EL device, an aromatic amine-based compound, for example, an amine derivative substituted with a heteroaryl ring may be used.

However, in an organic EL device using the material using the above-mentioned material, it may be is difficult provide a low driving voltage and sufficiently high emission efficiency. An organic EL device having a further lower driving voltage and further higher emission efficiency is desirable. Particularly, since the emission efficiency of the organic EL device in the blue emission region is low, improvement of the emission efficiency is desirable in the blue emission region when compared to the red emission region and the green emission region. To realize the driving at a low voltage and high emission efficiency of the organic EL device, the development of a novel material is desirable.

Embodiments advance the art by providing a material for an organic electroluminescent device driven at a low voltage and showing high emission efficiency particularly in a blue emission region and a green emission region, and an organic electroluminescent device including the same.

Embodiments provide a material for an organic EL device driven at a low voltage and having high emission efficiency and used in a layer of stacking layers disposed between an emission layer and an anode, and an organic EL device including the same.

For example, a material for an organic EL device according to an embodiment may maintain amorphous property and realize high mobility of holes by introducing a benzoxazinophenoxazine part with high planarity to the meta position of a phenylene group having small conjugation effect in an amine with high amorphous property and high hole mobility, and may realize the driving at a low voltage and high emission efficiency when used for the manufacture of an organic EL device. Remarkable effects may be obtained in a blue emission region.

According to embodiments, the organic EL device may maintain amorphous property and realize high mobility of holes by using a material introducing a benzoxazinophenoxazine part with high planarity to the meta position of a phenylene group having small conjugation effect in an amine with high amorphous property and high hole mobility in one layer of stacking layers disposed between an emission layer and an anode, and may realize the driving at a low voltage and high emission efficiency.

According to embodiments, a material for an organic EL device capable of being driven at a low voltage and having high emission efficiency, and an organic EL device including the same are provided. Particularly, a material for an organic EL device capable of being driven at a low voltage and having high emission efficiency and used in a layer of stacking layers between an emission layer and an anode in a blue emission region, and an organic EL device including the same are provided. In an amine having high amorphous property and high hole mobility, a benzoxazinophenoxazine part with high planarity is introduced to the meta position of a phenylene group with small conjugation effect, thereby maintaining amorphous property, realizing the high mobility of holes, a low driving voltage and high emission efficiency

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope thereof as set forth in the following claims.

What is claimed is:

1. A compound for an organic electroluminescent (EL) device, the compound being represented by the following General Formula (1), wherein X in General Formula (1) is selected from the following General Formulae (2), (3), and (4): 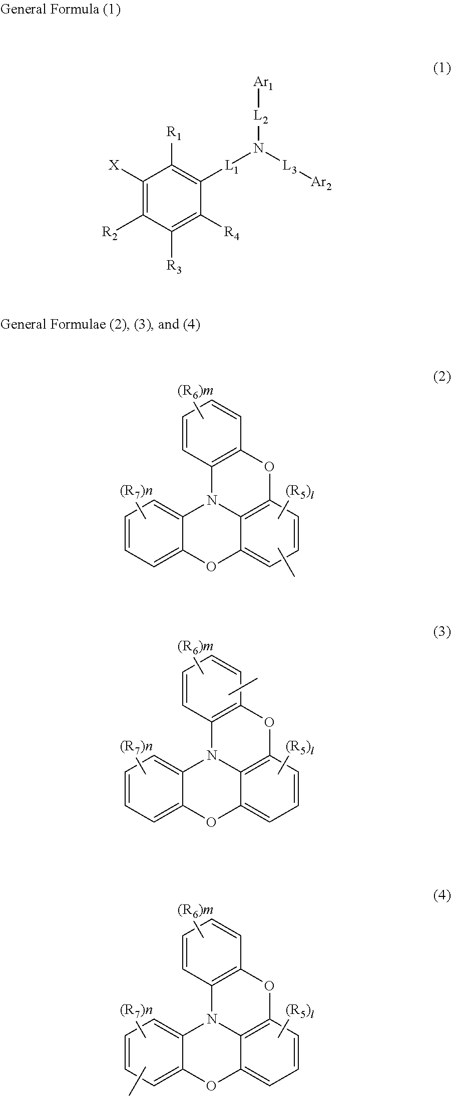
wherein, in General Formula (1),
R₁, R₂, R₃ and R₄ are independently a substituted or unsubstituted aryl group having 6 to 30 ring carbon atoms, a substituted or unsubstituted heteroaryl group having 1 to 30 ring carbon atoms, an alkyl group having 1 to 15 carbon atoms, a silyl group, a halogen atom, a hydrogen atom or a deuterium atom,
L₁, L₂ and L₃ are independently a divalent group selected from a substituted or unsubstituted alkylene group, aralkylene group, arylene group and heteroarylene group, or a direct linkage,
Ar₁ and Ar₂ are independently a substituted or unsubstituted aryl group having 6 to 30 ring carbon atoms or a substituted or unsubstituted heteroaryl group having 1 to 30 ring carbon atoms, and
in General Formulae (2), (3), and (4),
R₅, R₆ and R₇ are independently a substituted or unsubstituted aryl group having 6 to 30 ring carbon atoms, a substituted or unsubstituted heteroaryl group having 1 to 30 ring carbon atoms, an alkyl group having 1 to 15 carbon atoms, a silyl group, a halogen atom, a hydrogen atom or a deuterium atom, and
l, n and m are independently an integer from 0 to 2.

2. The compound for an organic EL device as claimed in claim 1, wherein X in General Formula (1) is represented by General Formula (2).

3. The compound for an organic EL device as claimed in claim 1, wherein Ar₁ and Ar₂ in the above General Formula (1) are a substituted or unsubstituted aryl group having 6 to 30 ring carbon atoms.

4. The compound for an organic EL device as claimed in claim 1, wherein multiple adjacent R₁, R₂, R₃ and R₄ and/or R₅, R₆ and R₇ are combined to form a saturated or unsaturated ring.

5. An organic electroluminescent (EL) device comprising a compound for an organic EL device in a layer of stacking layers between an emission layer and an anode,
wherein the compound for an organic EL device is represented by the following General Formula (1), and X in General Formula (1) is selected from the following General Formulae (2), (3) and (4): 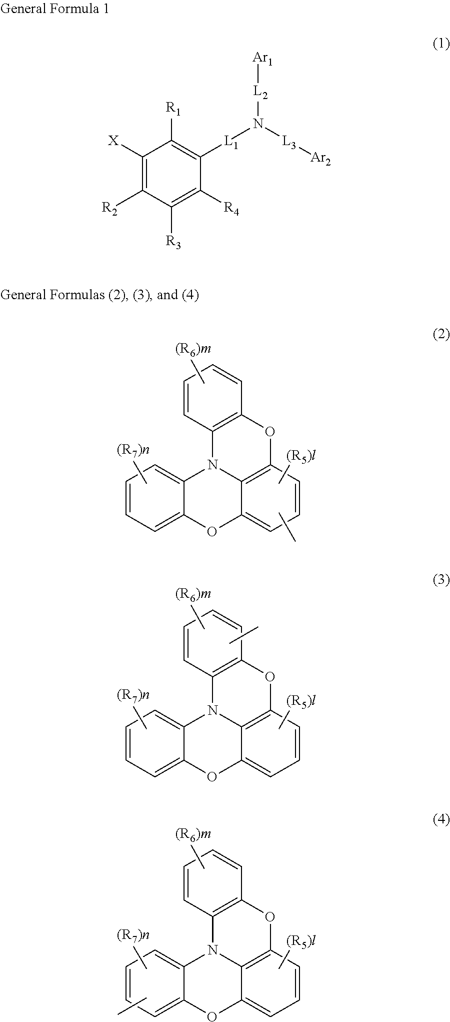
wherein, in General Formula (1),
R₁, R₂, R₃ and R₄ are independently a substituted or unsubstituted aryl group having 6 to 30 ring carbon atoms, a substituted or unsubstituted heteroaryl group having 1 to 30 ring carbon atoms, an alkyl group having 1 to 15 carbon atoms, a silyl group, a halogen atom, a hydrogen atom or a deuterium atom,
L₁, L₂ and L₃ are independently a divalent group selected from a substituted or unsubstituted alkylene group, aralkylene group, arylene group and heteroarylene group, or a direct linkage, and
Ar₁ and Ar₂ are independently a substituted or unsubstituted aryl group having 6 to 30 ring carbon atoms or a substituted or unsubstituted heteroaryl group having 1 to 30 ring carbon atoms, and
in General Formula (2), (3), and (4),
R₅, R₆ and R₇ are independently a substituted or unsubstituted aryl group having 6 to 30 ring carbon atoms, a substituted or unsubstituted heteroaryl group having 1 to 30 ring carbon atoms, an alkyl group having 1 to 15 carbon atoms, a silyl group, a halogen atom, a hydrogen atom or a deuterium atom, and
l, n and m are independently an integer from 0 to 2.

6. The organic EL device as claimed in claim 5, wherein X in General Formula (1) is represented by General Formula (2).

7. The organic EL device as claimed in claim 5, wherein Ar₁ and Ar₂ in the above General Formula (1) are a substituted or unsubstituted aryl group having 6 to 30 ring carbon atoms.

8. The organic EL device as claimed in claim 5, wherein multiple adjacent R₁, R₂, R₃ and R₄ and/or R₅, R₆ and R₇ are combined to form a saturated or unsaturated ring.

9. The organic EL device as claimed in claim 5, wherein the compound for an organic EL device is at least one of the following compounds 1 to 18:

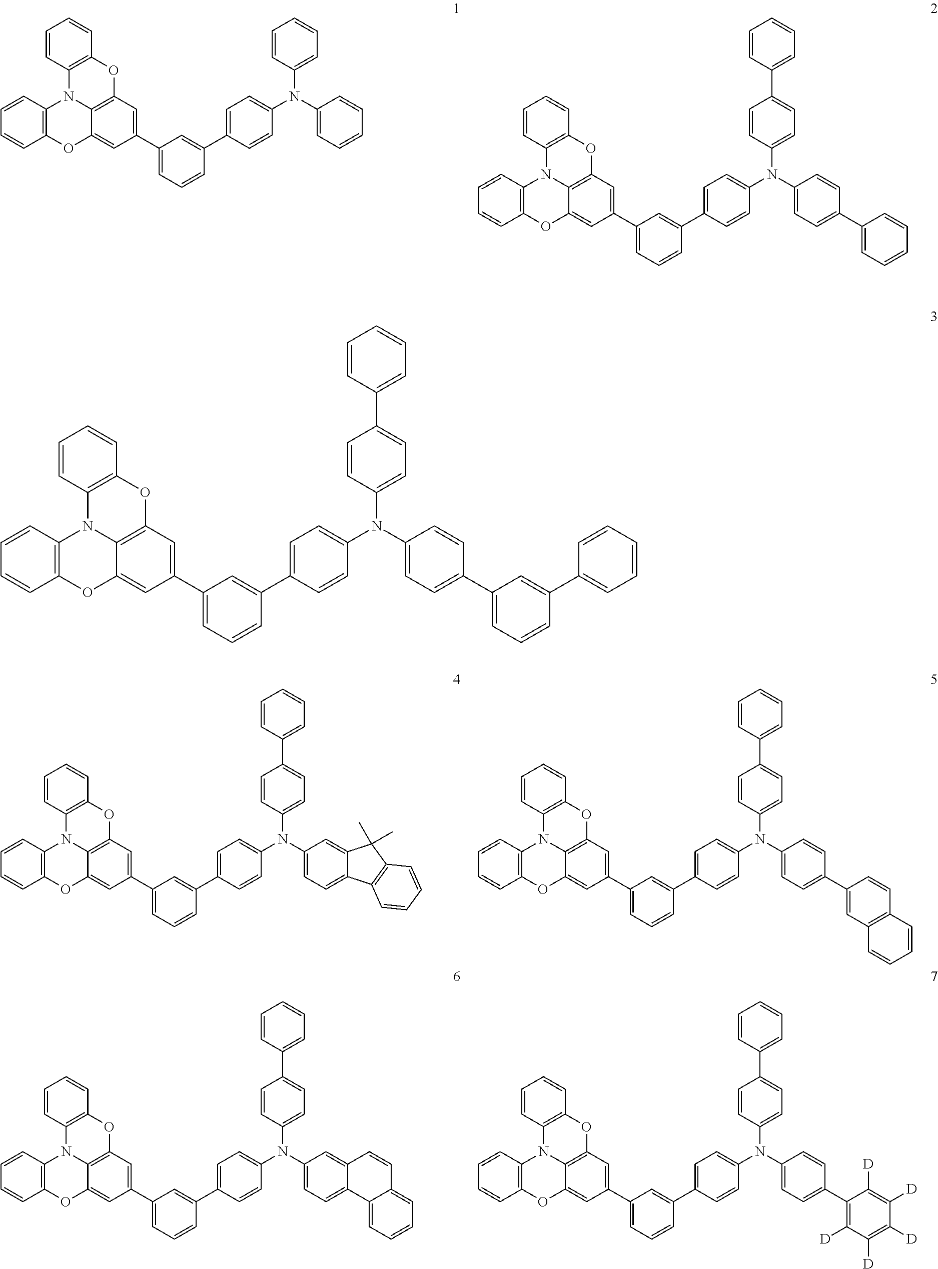

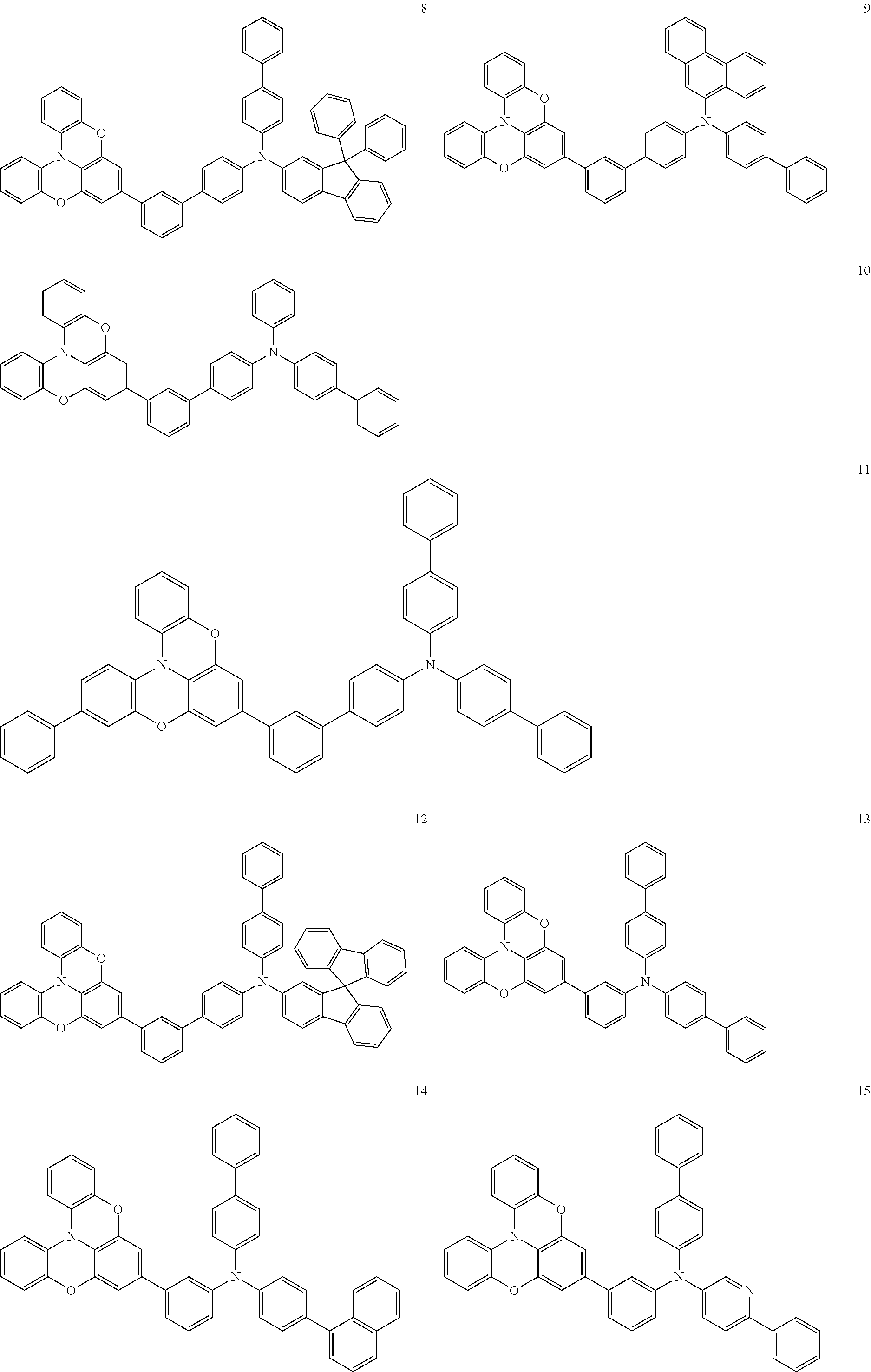

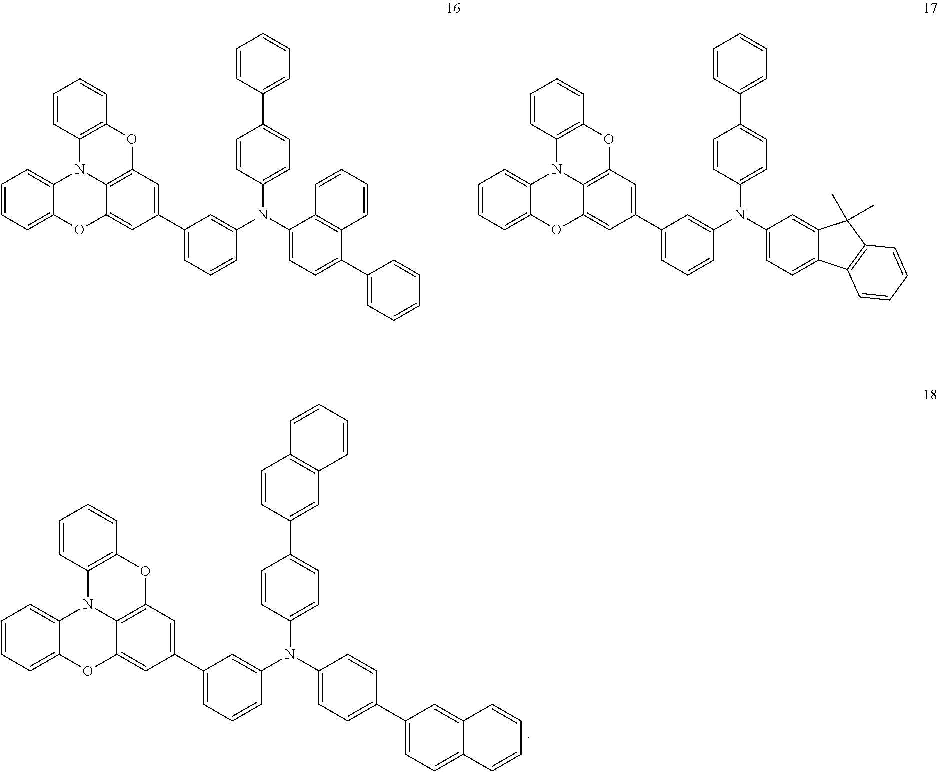 [SMILES: c1ccc(-c2ccc(N(c3cccc(-c4cc5c6c(c4)Oc4ccccc4N6c4ccccc4O5)c3)c3ccc(-c4ccccc4)c4ccccc34)cc2)cc1 ; CC1(C)c2ccccc2-c2ccc(N(c3ccc(-c4ccccc4)cc3)c3cccc(-c4cc5c6c(c4)Oc4ccccc4N6c4ccccc4O5)c3)cc21 ; c1cc(-c2cc3c4c(c2)Oc2ccccc2N4c2ccccc2O3)cc(N(c2ccc(-c3ccc4ccccc4c3)cc2)c2ccc(-c3ccc4ccccc4c3)cc2)c1]

10. The organic EL device of claim 5, wherein the compound for an organic EL device is at least one of the following compounds 19 to 36:

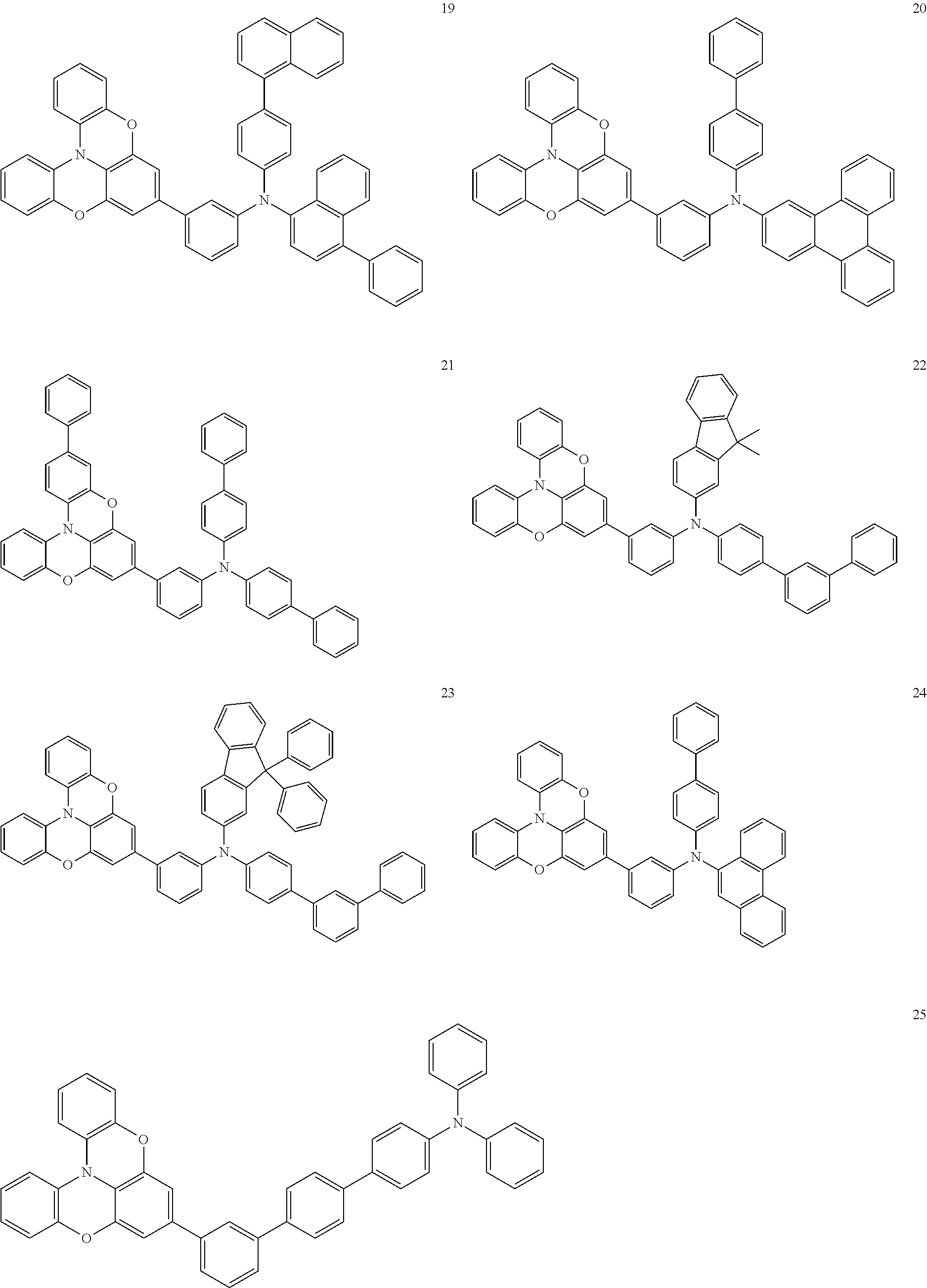

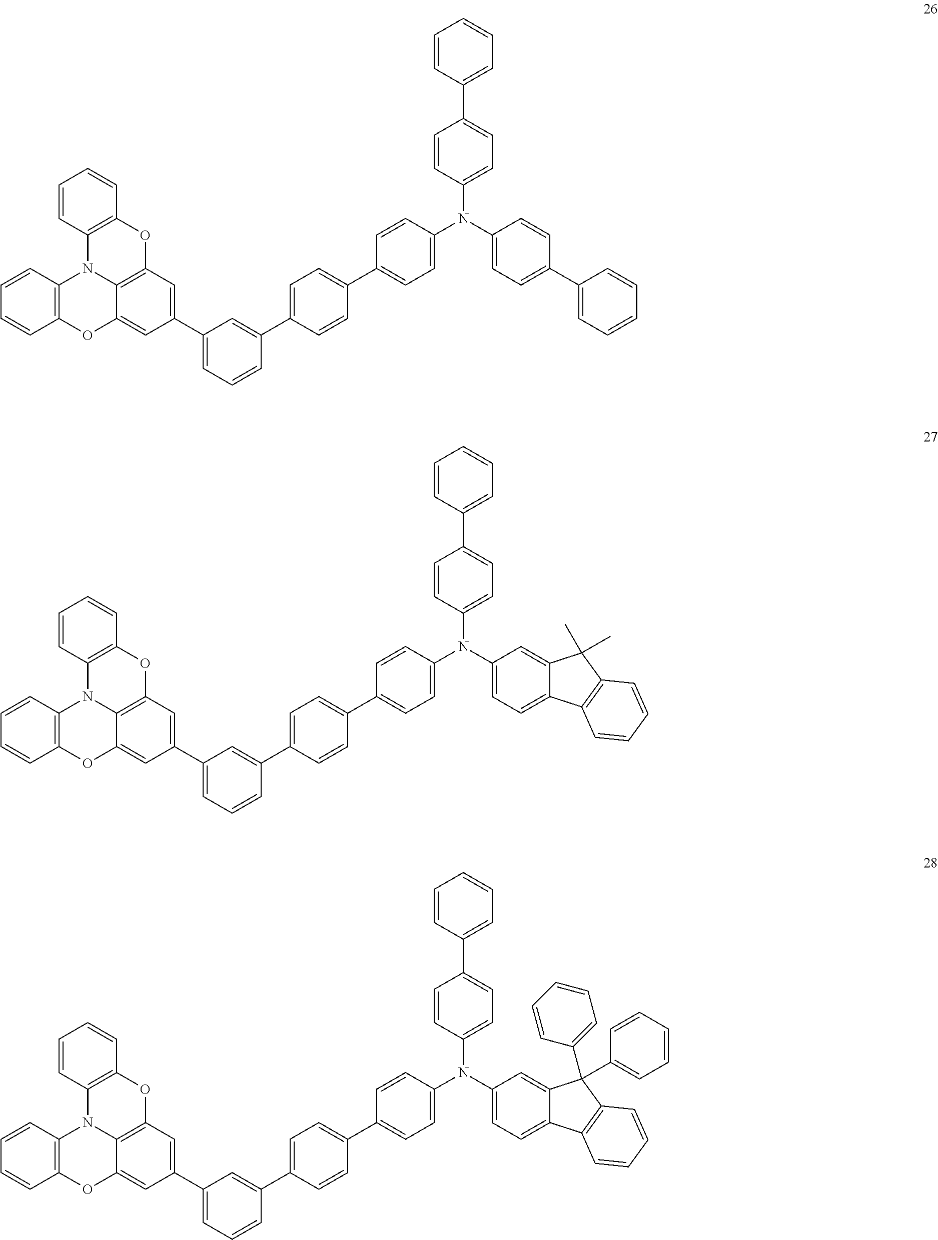 [SMILES: c1ccc(-c2ccc(N(c3ccc(-c4ccccc4)cc3)c3ccc(-c4ccc(-c5cccc(-c6cc7c8c(c6)Oc6ccccc6N8c6ccccc6O7)c5)cc4)cc3)cc2)cc1 ; CC1(C)c2ccccc2-c2ccc(N(c3ccc(-c4ccccc4)cc3)c3ccc(-c4ccc(-c5cccc(-c6cc7c8c(c6)Oc6ccccc6N8c6ccccc6O7)c5)cc4)cc3)cc21 ; c1ccc(-c2ccc(N(c3ccc(-c4ccc(-c5cccc(-c6cc7c8c(c6)Oc6ccccc6N8c6ccccc6O7)c5)cc4)cc3)c3ccc4c(c3)C(c3ccccc3)(c3ccccc3)c3ccccc3-4)cc2)cc1]

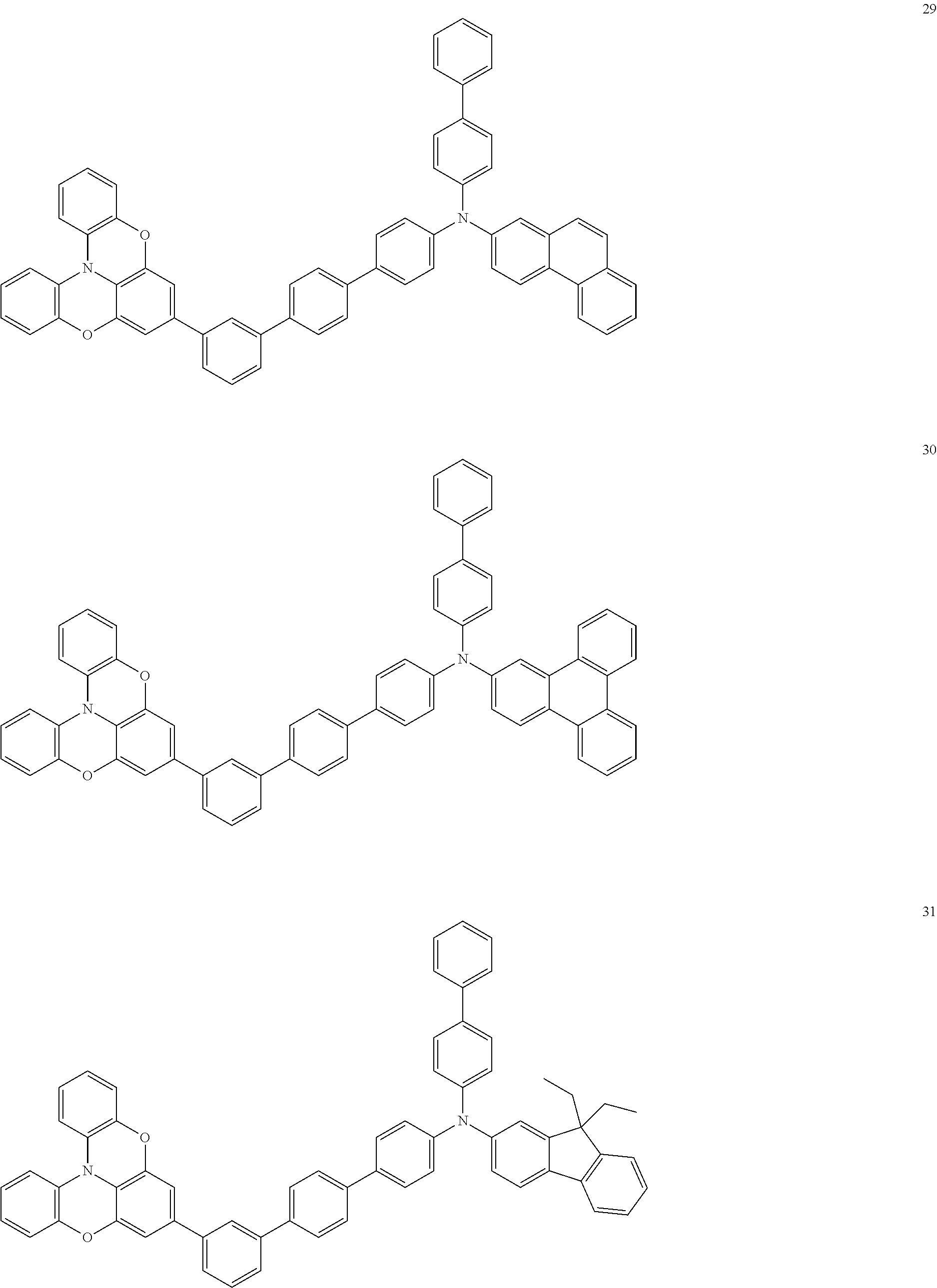 [SMILES: c1ccc(-c2ccc(N(c3ccc(-c4ccc(-c5cccc(-c6cc7c8c(c6)Oc6ccccc6N8c6ccccc6O7)c5)cc4)cc3)c3ccc4c(ccc5ccccc54)c3)cc2)cc1 ; c1ccc(-c2ccc(N(c3ccc(-c4ccc(-c5cccc(-c6cc7c8c(c6)Oc6ccccc6N8c6ccccc6O7)c5)cc4)cc3)c3ccc4c5ccccc5c5ccccc5c4c3)cc2)cc1 ; CCC1(CC)c2ccccc2-c2ccc(N(c3ccc(-c4ccccc4)cc3)c3ccc(-c4ccc(-c5cccc(-c6cc7c8c(c6)Oc6ccccc6N8c6ccccc6O7)c5)cc4)cc3)cc21]

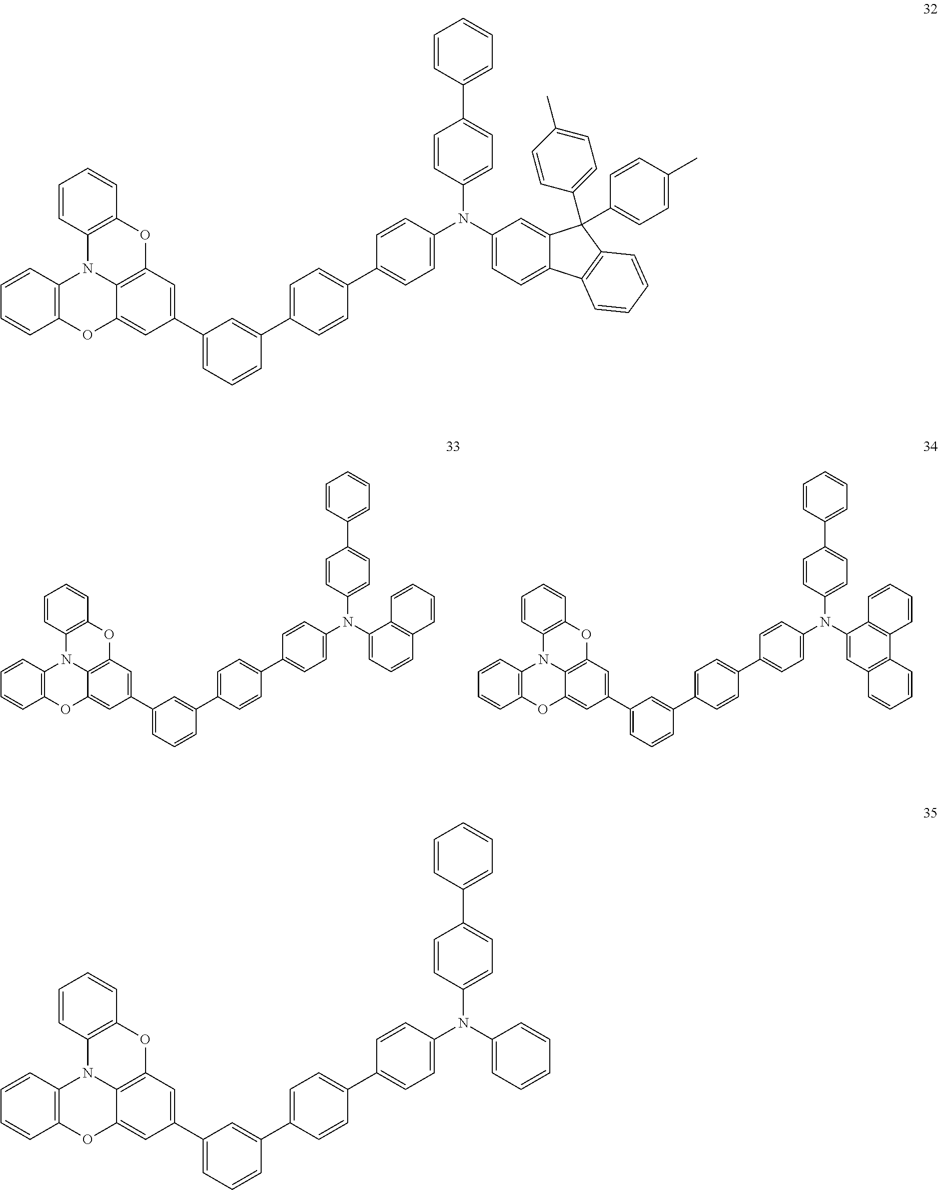 [SMILES: Cc1ccc(C2(c3ccc(C)cc3)c3ccccc3-c3ccc(N(c4ccc(-c5ccccc5)cc4)c4ccc(-c5ccc(-c6cccc(-c7cc8c9c(c7)Oc7ccccc7N9c7ccccc7O8)c6)cc5)cc4)cc32)cc1 ; c1ccc(-c2ccc(N(c3ccc(-c4ccc(-c5cccc(-c6cc7c8c(c6)Oc6ccccc6N8c6ccccc6O7)c5)cc4)cc3)c3cccc4ccccc34)cc2)cc1 ; c1ccc(-c2ccc(N(c3ccc(-c4ccc(-c5cccc(-c6cc7c8c(c6)Oc6ccccc6N8c6ccccc6O7)c5)cc4)cc3)c3cc4ccccc4c4ccccc34)cc2)cc1 ; c1ccc(-c2ccc(N(c3ccccc3)c3ccc(-c4ccc(-c5cccc(-c6cc7c8c(c6)Oc6ccccc6N8c6ccccc6O7)c5)cc4)cc3)cc2)cc1]

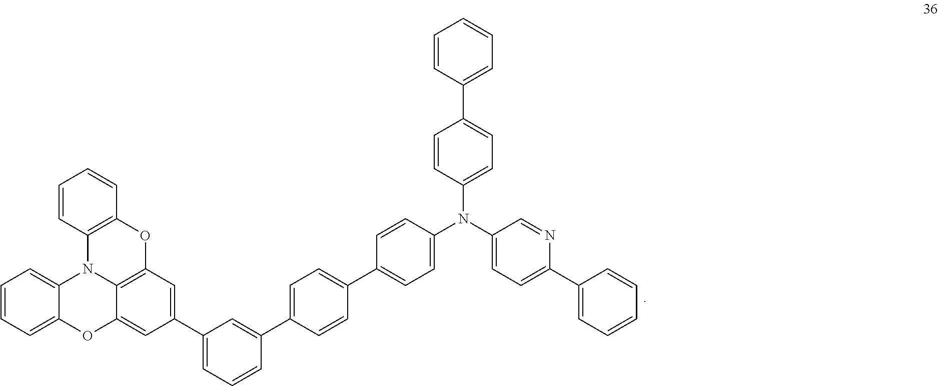 [SMILES: c1ccc(-c2ccc(N(c3ccc(-c4ccc(-c5cccc(-c6cc7c8c(c6)Oc6ccccc6N8c6ccccc6O7)c5)cc4)cc3)c3ccc(-c4ccccc4)nc3)cc2)cc1]

11. The organic EL device as claimed in claim 5, wherein the compound for an organic EL device is at least one of the following compounds 27 to 60: 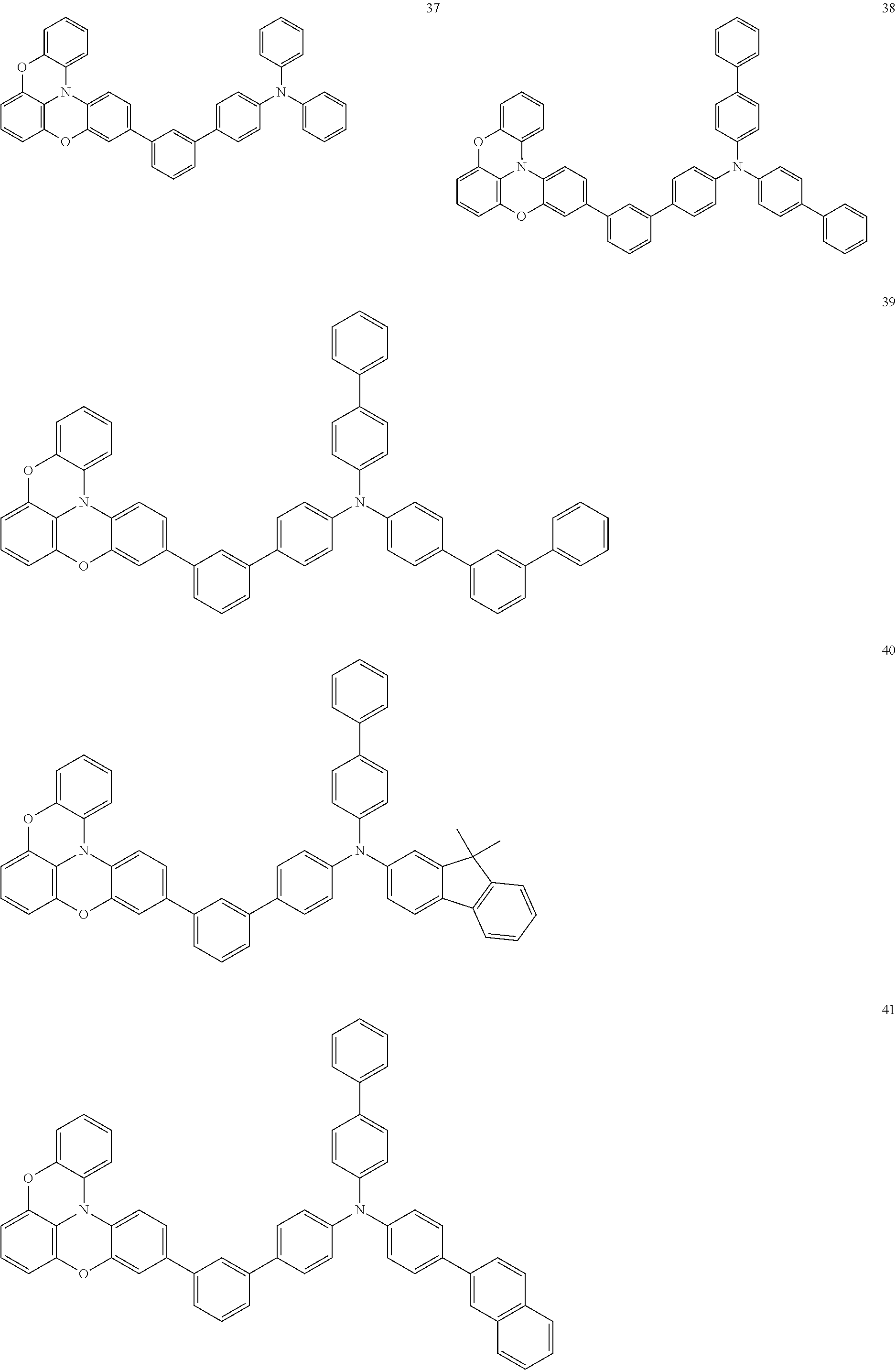 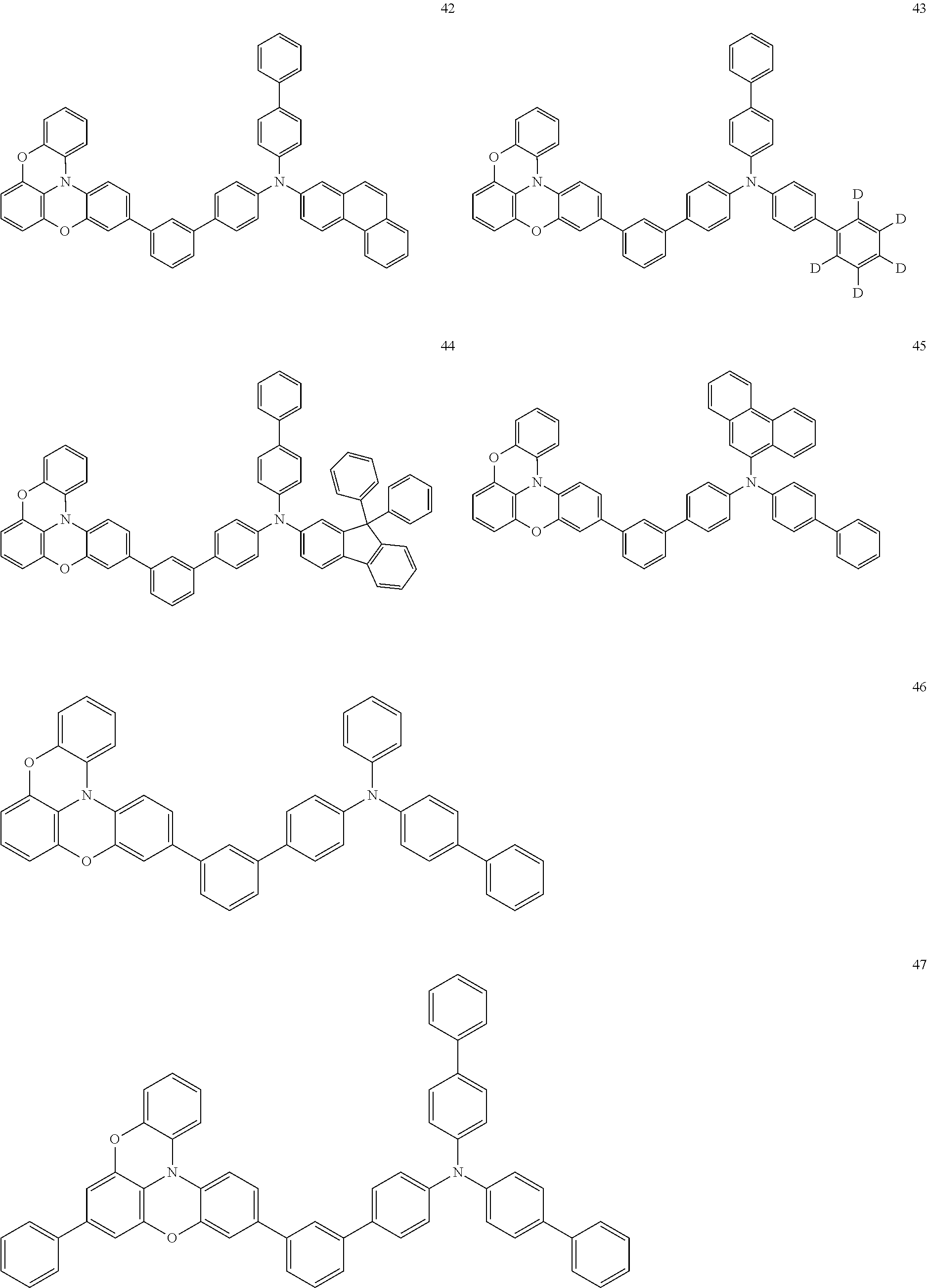 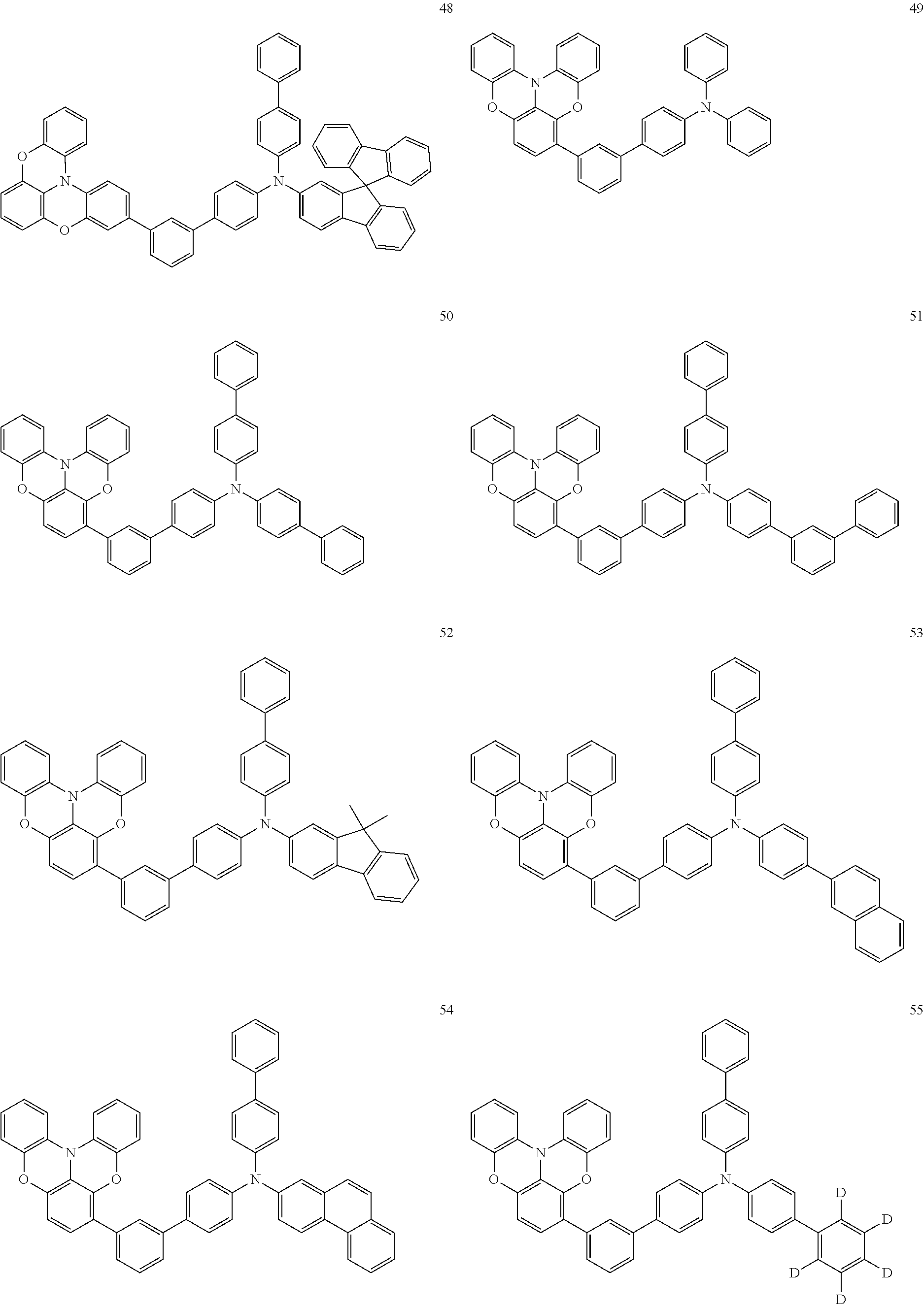 [chemical structure]
